(19) United States
(12) Patent Application Publication
(10) Pub. No.: US 2003/0180804 A1
(43) Pub. Date: Sep. 25, 2003

(54) Solid phase synthesis of chemical libraries

(75) Inventors: Kent E. Pryor, San Diego, CA (US); Ronald D. Lewis II, San Diego, CA (US); Jason W. Brown, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corvas International, Inc, San Diego, CA

(21) Appl. No.: 10/282,841

(22) Filed: Oct. 29, 2002

Related U.S. Application Data
(60) Provisional application No. 60/336,403, filed on Oct. 29, 2001.

(51) Int. Cl. G01N 33/53; C07D 333/42; G01N 33/543; C07D 207/30; C07D 307/02

(52) U.S. Cl. 435/7.1; 436/518; 548/561; 549/63; 549/491

(57) ABSTRACT

The present invention provides compounds and compound libraries that are useful as protease modulators. The compounds and compound libraries are preferably made using the methods of the present invention which utilize peptide synthesis and combinatorial chemistry methods on a solid phase.

### CROSS-REFERENCES TO RELATED APPLICATIONS

This application claims the benefit of United States Provisional Application No. 60/336,403, filed Oct. 29, 2001. The foregoing application is hereby incorporated by reference in its entirety for all purposes.

### FIELD OF THE INVENTION

The present invention provides compounds and compound libraries that are useful as protease modulators. The compounds and compound libraries are preferably made using the methods of the present invention which utilize peptide synthesis and combinatorial chemistry methods on a solid phase.

### BACKGROUND OF THE INVENTION

Combinatorial chemistry has become a powerful tool for drug discovery in the pharmaceutical and biotechnology industries. Generally speaking, combinatorial chemistry is defined as the repetitive and systematic covalent attachment of different structural moieties to one another to produce a mixture of numerous distinct molecular entities or target molecules (i.e., combinatorial libraries). Desired target molecules include for example, peptides, oligonucleotides, and small organic molecules. Frequently, combinatorial chemistry is utilized to generate a group of structurally related analogs, which can then be evaluated to establish structure-activity relationships (SAR) and to optimize biological efficacy.

Due to inherent versatility and high yields, there is great interest in the development of solid phase combinatorial synthesis methods. Solid phase methods are often employed to synthesize combinatorial libraries because they offer advantages over traditional solution-based methods. In solid phase methods, excess reagents and soluble by-products can be simply removed by resin washing. In addition, the techniques are readily amenable to parallelization, enabling many compounds to be prepared simultaneously. Moreover, resin-bound toxic compounds can be handled safely without risk to users or the environment. The combinatorial synthesis of peptide libraries is an especially important area of development.

†-P¹—(P²)_{y}—P⁴ I

In Formula I, P¹ is an arginine surrogate attached directly to a solid support; P² is an amino acid residue; y is an integer from 1 to about 12 wherein each P² can be the same or different; and P⁴ is selected from the group of R—, RC(O)—, RR¹NC(O)—, RSO₂—, RR¹N—SO₂—, RS(O)—, RR¹N—S(O)— and ROC(O)—, wherein R and R¹ are each independently selected from hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group.

In another embodiment, the present invention provides a combinatorial library comprising compounds of the formula:

P¹—(P²)_{y}—P⁴ Ia

In Formula Ia, P¹ is an arginine surrogate; P² is an amino acid residue; y is an integer from 1 to about 12 wherein each P² can be the same or different; and P⁴ is selected from the group of R—, RC(O)—, RR¹NC(O)—, RSO₂—, RR¹N—SO₂—, RS(O)—, RR¹N—S(O)— and ROC(O)—, wherein R and R¹ are each independently selected from hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group.

In yet another embodiment, the present invention provides a method for preparing a protease modulator having an arginine surrogate on a solid support comprising:

(a) attaching a protected arginine surrogate to the solid support to provide a protected support-bound arginine surrogate;

(b) deprotecting the support-bound arginine surrogate to form a deprotected support-bound arginine surrogate;

(c) contacting the deprotected support-bound arginine surrogate with at least one protected amino acid under conditions to provide a protected support-bound substituted arginine surrogate;

(d) deprotecting the protected support-bound substituted arginine surrogate to form a deprotected substituted support-bound arginine surrogate; and

(e) contacting the deprotected support-bound substituted arginine surrogate with at least one capping agent under conditions to provide a protease modulator having an arginine surrogate on the solid support. In certain aspects, the method further comprises

(f) removing the protease modulator from the solid support.

These and other embodiments and advantages will become more apparent when read with the accompanying drawings and detailed description of the invention, which follow.

### BRIEF DESCRIPTION OF THE DRAWINGS

FIGS. 1A-B illustrate a synthetic scheme of the present invention.

FIG. 2 illustrates one synthetic scheme to generate an arginine surrogate.

FIG. 3 illustrates an arginine surrogate attachment to and detachment from a solid support.

FIG. 4 illustrates various protecting groups useful in the present invention.

FIGS. 5A-C illustrate the degree of inhibition for the urokinase, endotheliase and matriptase enzymes while varying the P2 segment and the P3-P4 linkage type.

FIGS. 6A-L illustrate the degree of inhibition for the urokinase, endotheliase and matriptase enzymes while varying the P3 segment and holding constant the P3-P4 linkage.

FIGS. 7A-C illustrate the degree of inhibition for the urokinase, endotheliase and matriptase enzymes while varying the P4 segment.

### DETAILED DESCRIPTION OF THE INVENTION AND PREFERRED EMBODIMENTS

A. Definitions

As used in this disclosure, the following abbreviations and terms have the defined meaning, unless expressly modified in the context in which the term is used:

The term “Alloc” is allyloxycarbonyl.

The term “Alloc-Cl” is allyl chloroformate.

The term “BSA” means bovine serum albumin.

The term “Boc” is tert-butoxycarbonyl.

The term “Bom” is benzyloxymethyl.

The term “BOP” is benzotriazole-yl-oxy-tris-(dimethylamino)phosphonium hexafluorophosphate.

The term “Cbz” is benzyloxycarbonyl or carbobenzyloxy.

The term “2-Clz” is 2-chlorobenzyloxycarbonyl.

The term “DCM” is dichloromethane.

The term “DIEA” is N,N-diisopropylethylamine.

The term “DMF” is N,N-dimethylformamide.

The term “DMSO” is dimethylsulfoxide.

The term “EDC” is 1-ethyl-3-(3-dimethylaminopropyl)carbodiimide hydrochloride salt.

The term “Fmoc” is 9-fluorenylmethyloxycarbonyl.

The term “HATU” is O-(7-azabenzotriazol-1-yl-N,N,N′,N′,-tetramethyluronium hexafluorophosphate.

The term “HEPES” means (N-[2-hydroxyethyl]piperazine-N′-[4-butanesulfonic acid]).

The term “HF” is hydrogen fluoride.

The term “HOAT” or “HOAt” is 1-hydroxy-7-azabenzotriazole.

The terms “HOBT” or “HOBt” are 1-hydroxybenzotriazole monohydrate.

The term “HPLC” is high pressure liquid chromatography; high performance liquid chromatography.

The term “LAH” is lithium aluminum hydride.

The term “MS” is mass spectrometry.

The term “Mts” is mesitylene-2-sulphonyl.

The term “NMM” is N-methylmorpholine (also referred to as 4-methylmorpholine).

The term “NMR” is nuclear magnetic resonance spectroscopy.

The term “PG” is protecting group.

The term “PMC” is 2,2,5,7,8-pentamethylchroman-6-sulfonyl.

The term “PyBOP” is benzotriazole-yl-oxy-tris-pyrrolidino-phosphonium hexafluorophosphate.

The term “(Ph₃P)₄Pd” is tetrakis-(triphenylphosphine)palladium(0).

The terms “r.t.” or “RT” are room temperature.

The term “TBTU” is 2-(1H-benzotriazole-1-yl)-1,1,3,3-tetramethyluronium tetrafluoroborate.

The term “TeOC” is trimethylsilylethoxycarbonyl Me₃Si(CH₂)₂OCO.

The term “TFA” is trifluoroacetic acid.

The term “THF” is tetrahydrofuran.

The term “TLC” or “tlc” is thin layer chromatography.

The term “TFMSA” is trifluoromethylsulfonic acid, also commonly referred to as “triflic acid”.

The term “TMSOTf” is trimethylsilyltrifluoro methane sulfate.

The term “Tos” is p-toluenesulfonyl also referred to as Tosyl or Ts.

The term “Troc” is trichloroethoxycarbonyl (an amine protecting group removable with zinc).

NMR designations: s is singlet; d is doublet; m is multiplet; br is broad peak; t is triplet; and q is quartet.

The term “arginine surrogate” is a chemical moiety which functions like, or mimics the molecular interaction of an arginine residue and preferably exhibits reactivity similar to that of an arginine residue. Preferably, arginine surrogates of the present invention comprise an amidinyl or a guanidinyl functional group and have a molecular weight of about 60 to about 800. The arginine surrogate is the P¹ position as described herein.

For the purpose of the present invention, the term “combinatorial library” means a collection of molecules based upon a logical design and involving the selective combination of building blocks by means of iterative synthesis used to make the compounds described herein. Each molecular species in the library is referred to as a member of the library. The combinatorial library of the present invention represents a collection of molecules of sufficient number and diversity of design to afford a rich molecular population from which to identify biologically active members.

The darkened bead “●” represents a solid support that is stable in the presence of acids, bases and/or other reagents. A “solid support” is any form of bead, resin or the like, typically used in the art of peptide synthesis to provide a “handle” whereby a growing synthetic peptide chain can be made available for synthetic manipulation without the risk of loss in peptide yield typically experienced when such syntheses are conducted in solution; the terms “solid support” and “resin” are used interchangeably. The term “solid support” or, “support,” refer to a solid particulate, insoluble material to which a peptide, peptide analog, or peptidomimetic can be synthesized. Supports used in synthesizing peptides and peptide analogs are typically substantially inert and nonreactive with the reagents used in the synthesis of peptides and peptide analogs. Preferably, a solid support is a cross-linked resin, such as polystyrene. Preferred solid supports include, but are not limited to, 4-nitrophenyl carbonate resin, imidazole carbonate resin and succinimidyl carbonate resin.

The term “amino acid” refers to natural amino acids or unnatural amino acids, and amino acid analogs and their D and L stereoisomers if their structure allows such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). Unnatural amino acids include, but are not limited to, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2, 4-diaminoisobutyric acid, desmosine, 2,2′-diaminopimelic acid, 2,3-diaminopropionic acid, n-ethylglycine, N-ethylasparagine, hydroxylysine, all-hydroxylysine, 3-hydroxyproline, 4-hydroxyproline, isodesmosine, allo-isoleucine, N-methylglycine, N-methylisoleucine, N-methylvaline, norvaline (Nval), norleucine, omithine and pipecolic acid. Amino acid analogs include, but are not limited to, the natural and unnatural amino acids which are chemically blocked, reversibly or irreversibly, or modified on their N-terminal amino group or their side-chain groups, as for example, methionine sulfoxide, methionine sulfone (Met(O₂), S-(carboxymethyl)-cysteine, S-(carboxymethyl)-cysteine sulfoxide and S-(carboxymethyl)-cysteine sulfone, aspartic acid-(beta methyl ester), N-ethylglycine, and alanine carboxamide. Certain preferred amino acids include L-amino acids, D-amino acids, sarcosine (Sar), norvaline (Nval), β-alanine (β-Ala), cyclohexyl-glycine (chx-Gly), (tetrahydro-3-isoquinolinecarboxylic acid) (Tic), phenylglycine (phGly), 4-bromophenylalanine (4BrPhe), 3-fluorophenylalanine (3FPhe), and benzylserine Ser(Bz).

The term “amino acid residue” refers to radicals having the structure NH—R—C(O), wherein R typically is —CH (R*)—, wherein R* is H or a carbon containing substituent; or

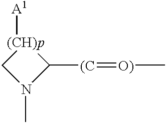

wherein A¹ is H, OH, lower alkyl of one to ten carbon atoms; alkenyl; nitro; cyano; halo; —S(O)q—, wherein, for purposes of this definition, q is 0,1, or 2; carboxylic acid or carboxylic acid derivatives, esters, amides, or aryl; and p is 1, 2, or 3, and when A¹is H, and p is 1, 2, or 3, the structure represents the azetidinecarboxylic acid, proline or pipecolic acid residues, respectively.

A peptide or peptide analog is a molecule comprising at least two amino acids or amino acid analogs linked through peptide (amide) linkage(s).

A peptidomimetic compound is any compound which structurally resembles or mimics a natural peptidyl array, and compounds comprising such residues; compounds which, although not a natural peptide, in the sense that it either contains no amino acids or contains amino acid analogs, exhibits biological activity similar to or expected of a peptidyl compound.

The terms “good leaving group” or “leaving group” are used herein to define a molecular substituent which, when used in conducting chemical syntheses, exhibits the desirable properties of being labile under defined synthetic conditions, and of being easily separated from synthetic products under defined conditions. Examples of such leaving groups include, but are not limited to, hydrogen, hydroxyl radicals, halogen atoms, p-nitrophenoxide, water, methyl groups, and the like.

The term “protecting group” is used herein to refer to well known moieties which have the desirable property of preventing specific chemical reactions at a site on a molecule undergoing chemical modification intended to be left unaffected by the particular chemical modification, while at the same time being easily removed from the molecule under conditions that do not adversely affect other sites in the modified molecule. Those skilled in the art have a wide variety of known protecting groups to choose from, depending on the nature of the chemical site to be protected. Reference is made, for example, to “Protective Groups in Organic Synthesis”, T. Greene (John Wiley & Sons, Inc., 1991), and to “Solid Phase Peptide Synthesis,” Stewart and Young (Pierce Chemical Co., 1984), herein incorporated by reference for this and other purposes. Examples of protecting groups known in the art include, but are not limited to, Cbz, Boc, Alloc, Fmoc, Troc, Teoc (Me₃Si (CH₂)₂OCO), PMC, and the like, and others disclosed herein.

The term “alkyl” refers to saturated aliphatic groups including straight-chain, branched-chain and cyclic groups.

The term “alkenyl” refers to unsaturated aliphatic groups having at least one double bond.

The term “aryl” refers to aromatic groups which have at least one ring having a conjugated pi electron system and includes carbocyclic aryl, heterocyclic aryl and biaryl groups, all of which can optionally be substituted with a substituent selected from, but not limited to, lower alkyl of one to ten carbon atoms; alkenyl; nitro; cyano; halo; —S(O)q—, wherein, for purposes of this definition, q is 0,1, or 2; carboxylic acid or carboxylic acid derivatives, esters, amides, hydroxyl, amines and the like.

The term “arylalkyl” refers to an alkyl group substituted with an aryl group. Exemplary arylalkyl groups include, but are not limited to, benzyl, picolyl, and the like, which can optionally be substituted with a substituent selected from, but not limited to, lower alkyl of one to ten carbon atoms; alkenyl; nitro; cyano; halo; S(O)q—, wherein, for purposes of this definition, q is 0, 1, or 2; carboxylic acid or carboxylic acid derivatives, esters, amides, hydroxyl, amines and the like.

The term “cycloalkyl” refers to an alkyl in which at least a portion of the molecule is in a closed ring configuration. Exemplary cycloalkyl groups include, but are not limited to, cyclohexyl, cyclopropyl, cyclobutyl, cyclopentyl and cycloheptyl.

The term “heterocycle” refers to any compound having a closed cyclic structure in which at least one atom thereof is other than a carbon atom. For example, cyclic alkyl, cyclic aryl, cyclic aralkyl compounds containing an optionally substituted nitrogen, an oxygen or a sulfur atom in the cyclic structure are heterocycles.

The term “heteroaryl” means a 5- or 6-membered heteroaromatic ring containing 1-4 heteroatoms selected from O, N and S; or a bicyclic 9- or 10-membered heteroaromatic ring system containing 1-4 heteroatoms selected from O, N and S; where the methine H atoms may be optionally substituted with alkyl, alkoxy or halogen. The 5- to 10-membered aromatic heterocyclic rings include, but are not limited to, imidazole, pyridine, indole, thiophene, benzopyranone, thiazole, furan, benzimidazole, quinoline, isoquinoline, quinoxaline, pyrimidine, pyrazine, tetrazole, pyrazole, benzoisoxazole and the like.

The term “heteroarylalkyl” means an alkyl containing a heteroaryl ring. For example: pyridinylmethyl, pyrimidinylethyl and the like.

The term “halo” or “halogen” refers to fluorine, chlorine, bromine and iodine.

The term “non-adverse conditions” describes conditions of reaction or synthesis which do not substantially adversely affect the skeleton of the peptide analog and/or its amino acid (and/or amino acid analog) components. One skilled in the art can readily identify functionalities, coupling procedures, deprotection procedures and cleavage conditions which meet these criteria.

Certain compounds of the present invention possess asymmetric carbon atoms (optical centers) or double bonds; the racemates, diastereomers, geometric isomers and individual isomers are all intended to be encompassed within the scope of the present invention. Optically active (R) and (S), or (D) and (L), isomers can be prepared using chiral synthons or chiral reagents, or resolved using conventional techniques. When the compounds described herein contain olefinic double bonds or other centers of geometric asymmetry, unless specified otherwise, it is intended to include both E and Z geometric isomers. Likewise, all tautomeric forms are intended to be included.

The term “modulate” or “modulation”, as used herein in its various forms, refers to the ability of a compound to activate or inhibit the function of a protease, either directly or indirectly. Modulation may occur in vitro or in vivo. Modulation, as described herein, is intended to encompass antagonism, agonism, partial antagonism and/or partial agonism of a function or characteristic associated with the protease of interest. The compounds of the present invention are protease modulating compounds.

B. Combinatorial Libraries

The present invention provides a combinatorial library having compounds of the formula:

●-P¹—(P²)_{y}—P⁴ I

wherein P¹, P², y, and P⁴ have been described above. The library can be used as a tool for drug discovery; i.e., as a means to discover novel lead compounds by screening the library against a variety of biological targets and to develop structure-activity relationship (SAR) data. In certain aspects, the compounds are useful as enzyme modulators such as protease inhibitors. As described herein, the inventive arginine surrogate (P¹) attached directly to a solid phase support is useful in the field of combinatorial chemistry and development of such chemical libraries. It will be appreciated that preferred methods within this aspect of the invention result in the development of combinatorial libraries having an arginine surrogate in the P¹ position (e.g., P⁴ P³ P² P¹).

Those skilled in the art will recognize that while a molecule bearing four residues is represented, peptides or peptide analogs of any desired length can be prepared according to the present methods by repeating the coupling steps as many times as necessary, in an iterative fashion (e.g., P⁴(P²)_{y}P¹, wherein y is about 2 to about 12). According to this aspect of the invention, a library is designed wherein the P¹ residue is kept constant while residues P², P³, and P⁴ are varied and incorporated into, for example, peptides and peptidomimetic organic compounds. In a preferred embodiment, multiple reactions are carried out in parallel. The P¹ site is first attached to the solid phase, followed by P², P³, P⁴, etc., thereby forming a library of peptide or peptidomimetic compounds available for structural-activity analysis in any number of in vitro or in vivo assay systems including protease modulation (e.g., inhibition) assays.

In one preferred embodiment, the arginine surrogates attached directly to a solid support has the formula:

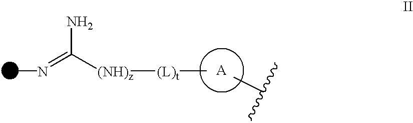

In Formula II, the ring ‘A’ is a five- or a six-membered optionally substituted cycloalkyl group, optionally substituted aryl group, or an optionally substituted heteroaryl group, wherein the heteroaryl group has from 1 to 3 heteroatoms selected from an optionally substituted N, O, and S; z is 0 or 1; L is selected from the group of alkylene optionally interrupted by a heteroatom (e.g., O or N) or, alternatively, L is sulfur; and t is 0 or 1.

In certain preferred aspects, the arginine surrogates attached directly to the solid support have the following formulae:

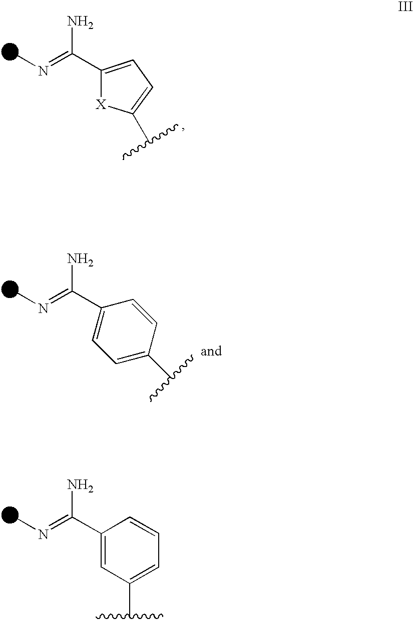

wherein X is a heteroatom selected from an optionally substituted N, S and O; preferably X is S.

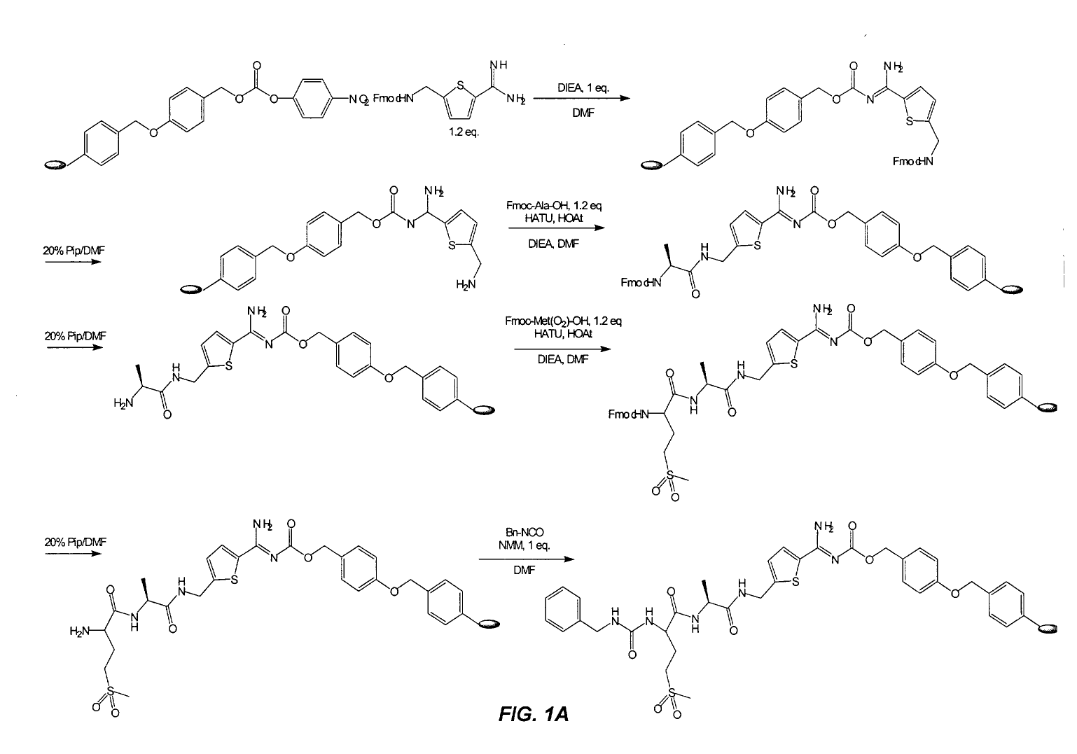

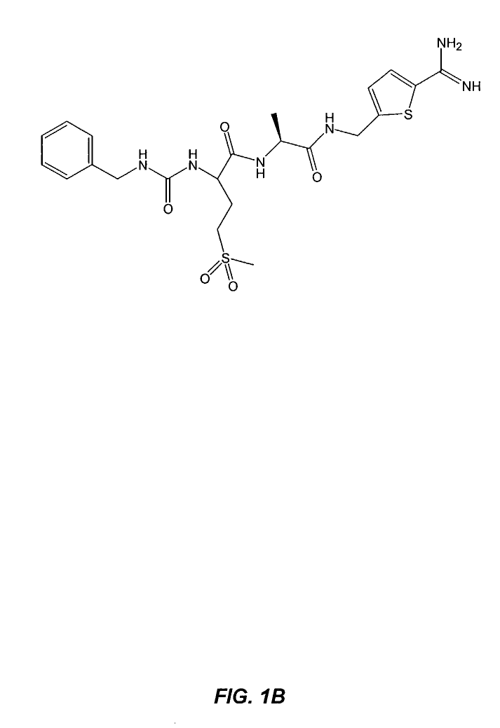

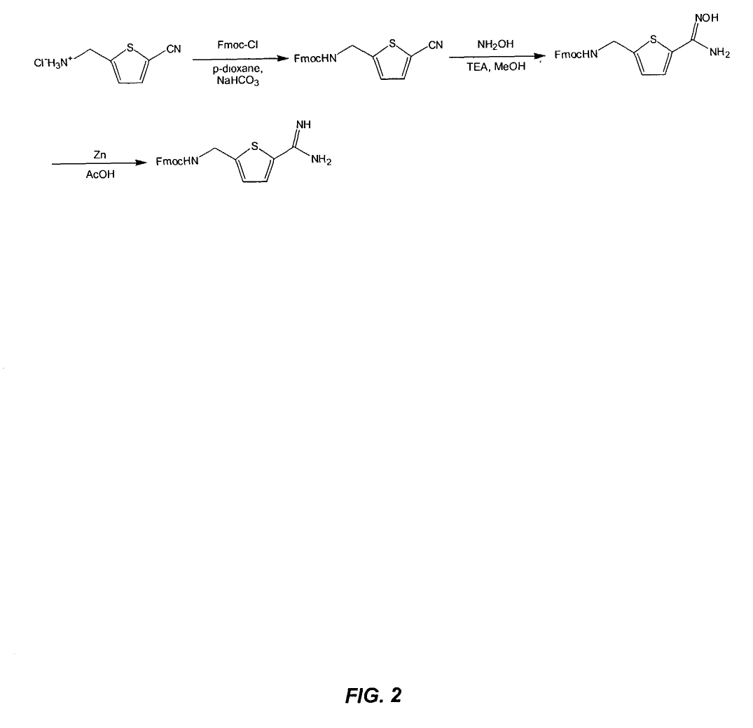

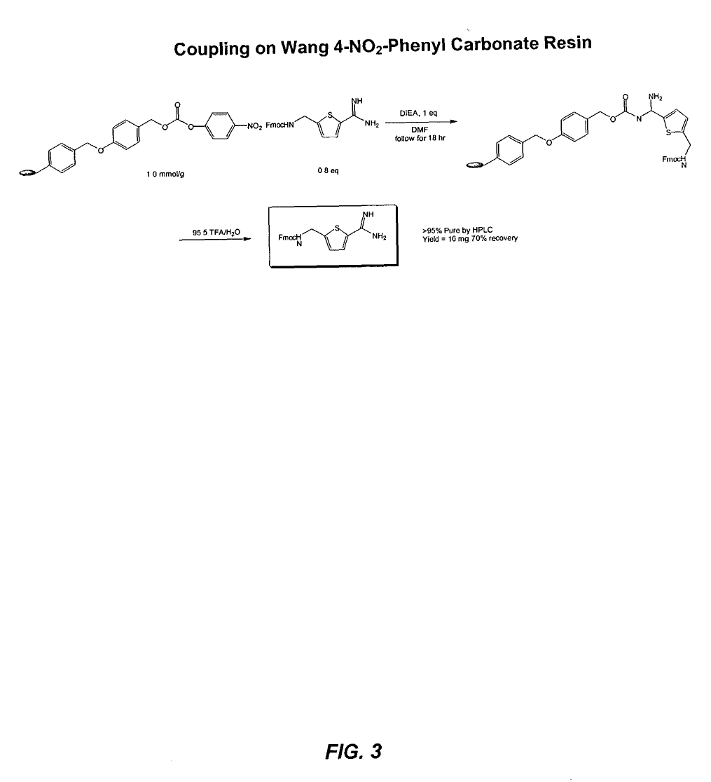

With reference to FIG. 1, in an especially preferred embodiment, the solid support comprises a 4-nitrophenyl carbonate functionality. In certain instances, treatment with an amidine functionality gives rise the following structure:

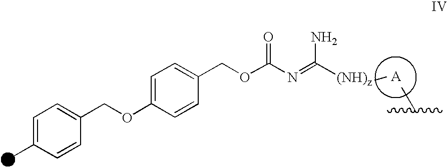

In one embodiment of Formula IV, the ring ‘A’ is defined as above. In a preferred aspect, the ring ‘A’ defines a 5-membered ring, as shown below in Formula V:

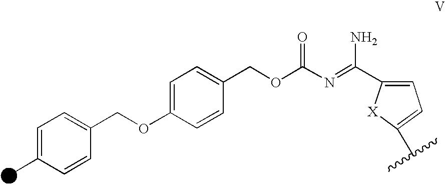

where X is a heteroatom selected from S, O and optionally substituted N; and preferably, X is S.

In certain other aspects, a compound of the combinatorial library can be defined as follows:

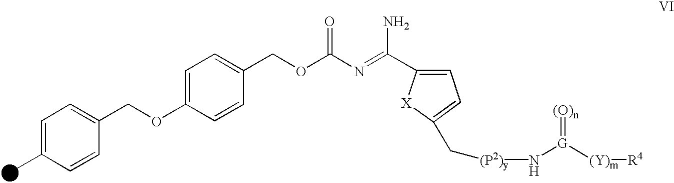

In one embodiment of Formula VI, X is a heteroatom selected from an optionally substituted N, S and O; each P² may be the same or a different amino acid residue; y is an integer of 1 to about 3; G is carbon and n is 1. Y is a heteroatom selected from the group of an optionally substituted N and O; m is 0 or 1; and R⁴is an optionally substituted aryl group, an optionally substituted heteroaryl group, an optionally substituted arylalkyl group, an optionally substituted heteroarylalkyl group, or an optionally substituted alkyl group. In an alternative embodiment, G is sulfur and n is 0, 1 or 2.

As will be appreciated by those of skill in the art, the process of library formation and parallel synthesis can be carried out in a number of formats. In one embodiment, the synthesis is conducted in a Whatman mini column, or the like, wherein standard peptide synthetic methods known in the art are used to extend the peptide chain, with each subsequent coupling being achieved at the carboxy terminus of each added residue. Following synthesis, the multiple peptide variants are cleaved from the resin, isolated, and tested for biological activity. Likewise, using the arginine surrogate of the present invention, automated synthesis of a library of peptides or peptide analogs can be conducted in any commercially available peptide synthesizer.

C. Methods

Methods for the synthesis of large numbers of diverse compounds that can be screened for various possible physiological or other activities are advantageous. Techniques have been developed in which individual units are added sequentially as part of the chemical synthesis to produce all, or a substantial number, of all the possible compounds which can result from all the different choices possible at each sequential stage of the synthesis. Many diverse compounds are produced by a series of reactions of a multiplicity of synthons in various combinations. Each compound in a combinatorial library results from the reaction of a subset of synthons.

As such, in another aspect, the present invention provides a method for preparing an enzyme modulator (e.g., protease inhibitor) modulator having an arginine surrogate on a solid support, comprising:

(a) attaching a protected arginine surrogate to the a solid support to provide a protected support-bound arginine surrogate;

(b) deprotecting the support-bound arginine surrogate to form a deprotected support-bound arginine surrogate;

(c) contacting the deprotected support-bound arginine surrogate with at least one protected amino acid under conditions to provide a protected support-bound substituted arginine surrogate;

(d) deprotecting the protected support-bound substituted arginine surrogate to form a deprotected support-bound substituted arginine surrogate; and

(e) contacting the deprotected support-bound substituted arginine surrogate with at least one capping agent (i.e., P⁴) under conditions to provide an enzyme modulator having an arginine surrogate on the solid support. In certain aspects, the method further comprises

(f) removing the protease modulator from the solid support.

In certain preferred aspects, the method further comprises repeating step (c) and step (d) in iterative fashion with about 2 to about 12 protected amino acids. Optionally, the protease modulator having an arginine surrogate is removed from the solid support.

In one preferred embodiment, the protected arginine surrogate has the formula:

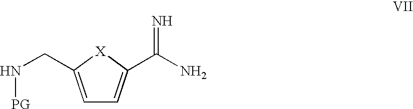

In Formula VII is a heteroatom selected from optionally substituted N, S and O; and PG is a protecting group (e.g., Fmoc, Alloc and Teoc).

Standard peptide synthetic methods can be employed in this process, subsequent to the initial formation of the arginine surrogate directly attached to the solid phase. In certain aspects, after cleavage from the solid phase, the synthesized library of peptides or peptide analogs bearing the original arginine surrogate as the P¹ residue can then be optionally purified and tested for biological activity.

It will be appreciated that where reactive moieties are present, as in reactive amino acid side-chains, methods are known for protecting such sites. Accordingly, where a reactive heteroatom exists in an amino acid side-chain, or where reaction at an amino acid or amino acid analogs' amino-terminal nitrogen is to be avoided, the following guidelines and procedures will apply. However, where side-chains are non-reactive, those skilled in the art will appreciate that protection is not required (e.g., methylcystine is already effectively protected; the phenylalanine side-chain is relatively non-reactive). In particular, with regard to an arginine moiety, wherein the side-chain has a guanidino group (the arginine side-chain is: —(CH₂)₃—NH—(C═NH₂⁺)—NH₂, wherein the moiety —NH—(C═NH₂⁺)—NH₂ represents the guanidino functionality), single or double protection of the side-chain may be desirable in order to achieve a desired reaction at another site, e.g., at the amino-terminal nitrogen, without production of a plurality of unwanted side-chain reactions.

In practicing the methods of the present invention, the following considerations apply to the selection of α-amino protecting groups, omega side-chain protecting groups, and carboxy protecting groups. In selecting suitable α-amino protecting groups (PG1) to be used during the synthesis of compounds of this invention the α-amino protecting group should:

(i) render the α-amino function inert under the conditions employed in the coupling reaction;

(ii) be readily removable after the coupling reaction under conditions that will not remove side-chain or carboxy terminus protecting groups; and

(iii) eliminate the possibility of racemization upon activation prior to coupling.

A suitable α-amino protecting groups are preferably stable to mildly basic coupling conditions.

(a) For example, amides prepared from acid chlorides or anhydrides are stable to acidic or basic hydrolysis. Suitable amide protecting groups include, but are not limited to, formamide, acetamide, chloroacetamide, trichloroacetamide, trifluoroacetamide and phenylacetamide. Among simple amides, hydrolytic stability increases from formyl, to acetyl to benzoyl. In most instances, the amide protecting groups are hydrolyzed by heating using strong acid or base conditions.

(b) Benzyloxycarbonyl (CBz), 2-chlorobenzyloxycarbonyl (2-Clz), cycloalkyloxycarbonyl, and isopropyloxycarbonyl require strong acids, such as hydrogen fluoride, hydrogen bromide or boron trifluoroacetate in trifluoroacetic acid for their removal.

The CBz and the 2-Clz groups can most conveniently be cleaved by hydrogenation over palladium on carbon in methanol. A suitable α-amino protecting group includes for instance, fluorenylmethyloxycarbonyl (Fmoc), which can be cleaved by using 20% piperidine/DMF or excess diethylamine in THF. Allyloxycarbonyl (Alloc) can be cleaved by Pd (0) catalyst transfer of the allyl group to an acceptor nucleophile such as morpholine, dimedone, tributyl tin hydride and N-methyl aniline. Preferred α-amino protecting groups (PG) include, but are not limited to, Fmoc, Alloc, and Cbz.

An amino acid side-chain protecting group should:

(i) render the protected side-chain functional group inert under the conditions employed in the coupling reaction;

(ii) be stable under the conditions employed in removing the α-amino or the carboxy terminus protecting groups, and

(iii) be readily removable upon completion of the desired peptide under reaction conditions that will not alter the structure of the peptide chain.

A suitable amino acid side-chain protecting group may be selected from the group consisting of (methods for cleavage of these protecting groups are shown in brackets [ ]):

(a) for protection of lysine amino groups, any of the groups mentioned above for the protection of α-amino protecting groups are suitable.

(b) for protection of arginine guanidino group, the preferred protecting groups include, but are not limited to, nitro [H₂/Pd/C, HF], benzyloxycarbonyl (CBz) [HF, TFMSA, TMSOTf, H₂/Pd/C], tert-butyloxycarbonyl (Boc) [TFA], 2,2,5,7,8-pentamethylchroman-6-sulfonyl (Pmc) [TFA], 2,3,6-trimethyl-4-methoxyphenylsulfonyl (Mtr) [TFA], p-toluenesulfonyl (Tos) [HF, TFMSA], mesitylene-2-sulphonyl (Mts) [HF, TFMSA], allyloxycarbonyl (Alloc) [Pd(0), morpholine].

(c) For protection of serine and threonine hydroxyl groups, protecting groups include, but are not limited to, trityl [1% TFA], tert-butyl [TFA], benzyl, and substituted benzyl groups such as 4-methoxybenzyl, 4-chlorobenzyl, 2-chlorobenzyl, and 2,6-dichlorobenzyl which are cleaved by a similar method [HF, TFMSA, H₂/Pd/C].

(d) For protection of the tyrosine phenolic group, protecting groups such as tert-butyl [TFA], trityl [1% TFA], benzyl, 2-bromobenzyl and 2,6-dichlorobenzyl, all cleaved by the same reagents [HF, TFMSA, H₂/Pd/C], are suitably employed.

(e) For protection of aspartic and glutamic acid side-chains carboxy groups, protecting groups include methyl [OH⁻, H⁺], ethyl [OH⁻, H⁺], t-butyl [TFA], allyl [Pd (0), morpholine], cyclohexyl [HF, TMSOTf], or benzyl groups [HF, TFMSA, TMSOTf, H₂/Pd/C].

(f) For protection of asparagine and glutamine side-chains, protecting groups include trityl [TFA] and xanthyl [TFA].

(g) For protection of the histidine imidazole group, suitable protecting groups include 2,4-dinitrophenyl (Dnp) [thiophenol], trityl [TFA], benzyloxymethyl (Bom) [HF, TFMSA, TMSOTf, H₂/Pd/C], p-toluene sulfonyl (Tos) [HF, TFMSA], and benzyloxycarbonyl (Cbz) [HF, H₂/Pd/C].

(h) For protection of cysteine sulfhydryl group, suitable protecting groups include trityl [TFA], 4-methylbenzyl (pMeBzl) [HF, TFMSA], 4-methoxybenzyl (pMeOBzl) [HF, TFMSA], acetamidomethyl (Acm) [H₂, Hg²⁺], tert-Butyl (tBu) [Hg²⁺].

(i) For protection of tryptophan indole group, suitable protecting groups include formyl [10% piperidine in DMF, followed by HF] and tert-butyloxycarbonyl (Boc) [TFA]. A carboxy terminus protecting group should:

(i) render the protected functional group inert under the conditions employed in the coupling reaction,

(ii) be stable under the conditions employed in removing the α-amino or the side-chain protecting groups, and

(iii) be readily removable upon completion of the desired peptide under reaction conditions that will not alter the structure of the peptide chain. For the protection of the carboxy terminus of amino acids suitable protecting groups include the esters methyl [OH—, H+], ethyl [OH—, H+], tert-butyl [TFA], benzyl [OH—, H₂/Pd/C] and allyl [Pd(0), morpholine] groups.

The materials upon which the combinatorial syntheses of the present invention are performed are referred to as solid supports, beads, solid phase and resins. These terms are intended to include, but are not limited to, beads, pellets, disks, fibers, gels, or particles such as cellulose beads, pore-glass beads, silica gels, polystyrene beads optionally cross-linked with divinylbenzene and optionally grafted with polyethylene glycol and optionally functionalized with amino, hydroxy, carboxy, or halo groups, grafted co-poly beads, polyacrylamide beads, latex beads, dimethylacrylamide beads optionally cross-linked with N,N′-bis-acryloyl ethylene diamine, glass particles coated with hydrophobic polymer, etc., i.e., material having a rigid or semi-rigid surface; and soluble supports such as low molecular weight non-cross-linked polystyrene.

D. Cleavage Conditions

Standard methods for cleavage of the peptide arginine surrogates or peptide analogs prepared according to the method of this invention can be used. Standard cleavage conditions include, for example, treatment with HF; TFA; TFA:H₂O (e.g., 9:1); (93:5:2 TFA/H₂O/TIS); 95:5 TFA/H₂O; and TFA:DCM:H₂O (e.g., 5:4:1), all result in good yields.

Variations of these conditions, as in minor variations in the time or solvent ratios, or use of solvents or acids having similar properties, come within the scope of this aspect of the invention.

E. Enzyme Modulators

Another aspect of the present invention is the use of the combinatorial library of Formulae I or Ia in assays to discover biologically active compounds or ligands. Thus another aspect of the invention is a method for identifying a compound having a desired characteristic, which comprises synthesizing a combinatorial library of Formulae I or Ia and testing the library, either attached to (i.e. Formula I) or detached from (i.e. Formula Ia) the solid supports, in an assay which identifies compounds having the desired characteristic.

In certain embodiments, the present invention provides a protease modulator having the formula:

P¹—(P²)_{y}—P⁴ Ia

wherein: P¹, P², y and P⁴ have previously been described. In the field of protease inhibition, a number of peptide and peptide analog inhibitors have been developed, as have methods for their synthesis (see for example, U.S. Pat. Nos. 5,283,293; 5,367,072; 5,371,072; 5,492,895; 5,514,777; 5,597,804; 5,637,599; 5,646,165; 5,656,600; 5,656,645; 5,681,844; 5,696,231; 5,703,208; 5,714,580; 5,731,413; 5,739,112; all of which are herein incorporated by reference for their disclosure of known protease inhibitors and methods of synthesis and use thereof).

In certain other embodiments, the present invention provides a protease modulator having the formula:

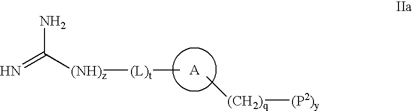

wherein the ring ‘A’, z, L, t, q and y have previously been described.

In yet another aspect, the present invention provides a protease modulator having the formula

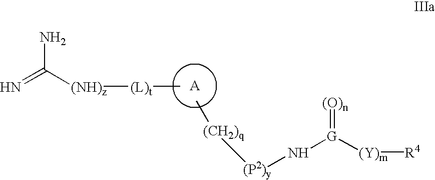

wherein the ring ‘A’ is a member selected from the group of a five- or a six-membered optionally substituted cycloalkyl group, optionally substituted aryl group and an optionally substituted heteroaryl group, wherein the heteroaryl group has from 1 to 3 heteroatoms selected from the group of N, O, and S. In Formula IIIa, each P² may be the same or a different amino acid residue; y is an integer of 1 to about 3; G is carbon and n is 1; or alternatively, G is sulfur and n is 0, 1 or 2; Y is a heteroatom selected from the group of optionally substituted N and O; m is 0 or 1; and R⁴ is an optionally substituted aryl group, an optionally substituted heteroaryl group, an optionally substituted arylalkyl group, an optionally substituted heteroarylalkyl group and an optionally substituted alkyl group.

In the protease modulators of the present invention, the ring ‘A’ in formula IIIa is preferably a thiophene ring, thus having the formula

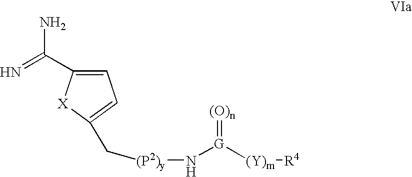

wherein X, P², y, G, Y m and R⁴ have been previously described.

Preferred enzyme modulators of Formula Ia wherein y=2, are set forth in Table I.

TABLE I

| | P1 | P2 | P3 | P4 |
|---|---|---|---|---|
| 1 | 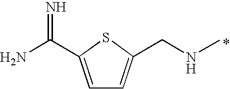 | 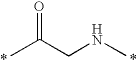 | 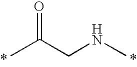 | 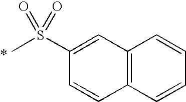 |
| 2 | | 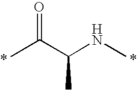 | 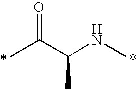 | 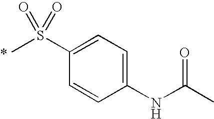 |
| 3 | | 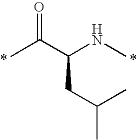 | 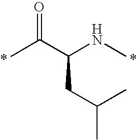 | 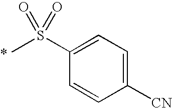 |
| 4 | | 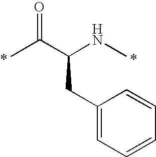 | 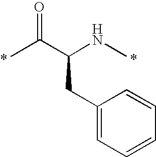 | 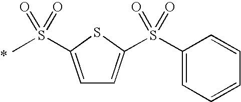 |
| 5 | | 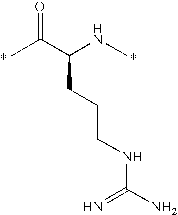 | 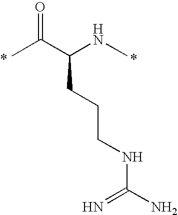 | 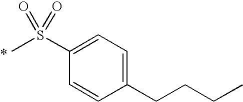 |
| 6 | | 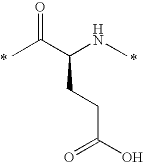 | 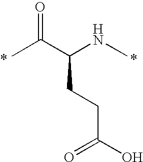 | 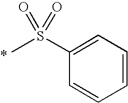 |
| 7 | | 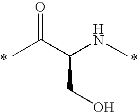 | 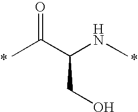 | 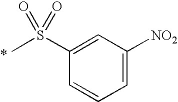 |
| 8 | | 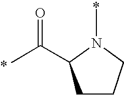 | 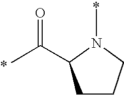 | 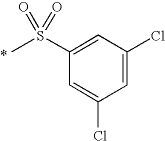 |
| 9 | | 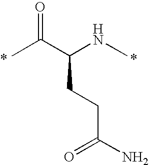 | 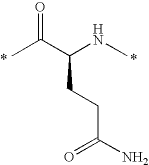 | 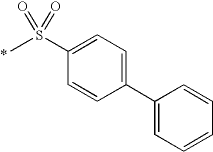 |
| 10 | | 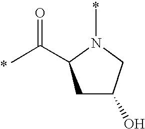 | 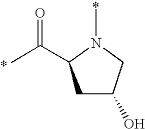 | 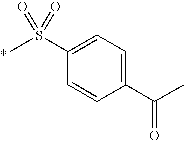 |
| 11 | | | 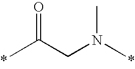 | 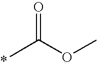 |
| 12 | | | 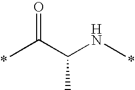 | 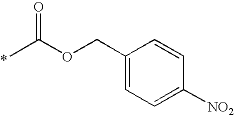 |
| 13 | | | 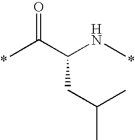 | 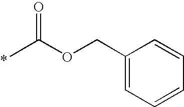 |
| 14 | | | 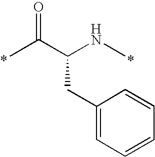 | 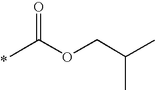 |
| 15 | | | 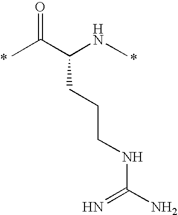 | 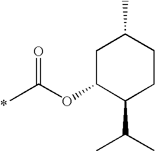 |
| 16 | | | 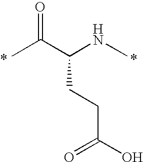 | 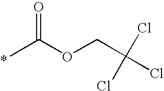 |
| 17 | | | 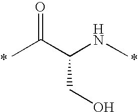 | 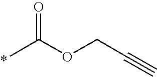 |
| 18 | | | 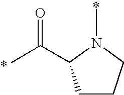 | 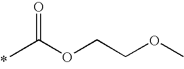 |
| 19 | | | 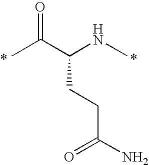 | 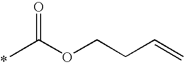 |
| 20 | | | 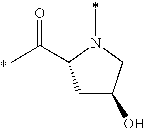 | 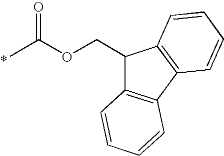 |
| 21 | | | 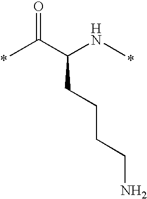 | 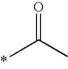 |
| 22 | | | 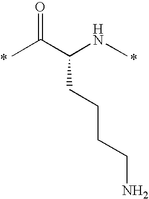 | 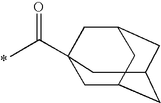 |
| 23 | | | 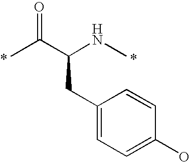 | 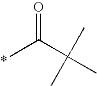 |
| 24 | | | 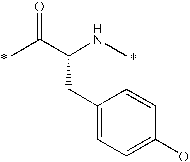 | 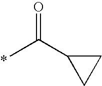 |
| 25 | | | 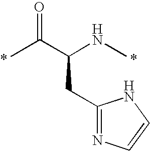 | 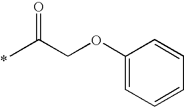 |
| 26 | | | 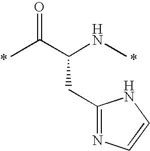 | 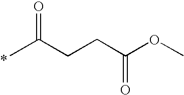 |
| 27 | | | 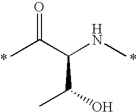 | 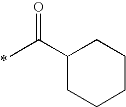 |
| 28 | | | 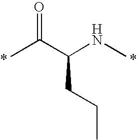 | 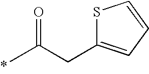 |
| 29 | | | 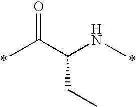 | 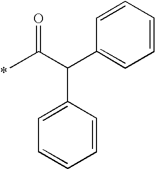 |
| 30 | | | 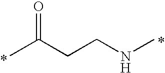 | 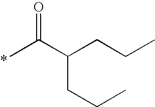 |
| 31 | | | 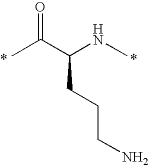 | 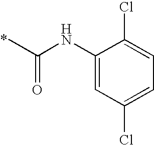 |
| 32 | | | 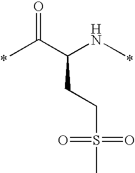 | 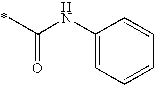 |
| 33 | | | 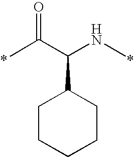 | 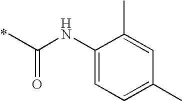 |
| 34 | | | 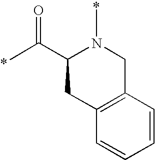 | 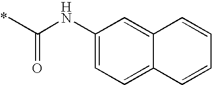 |
| 35 | | | 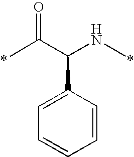 | 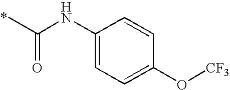 |
| 36 | | | 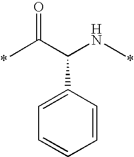 | 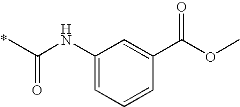 |
| 37 | | | 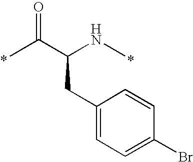 | 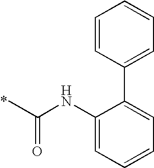 |
| 38 | | | 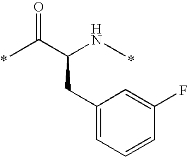 | 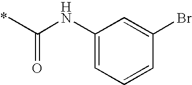 |
| 39 | | | 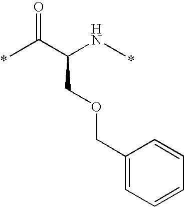 | 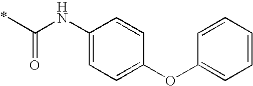 |
| 40 | | | 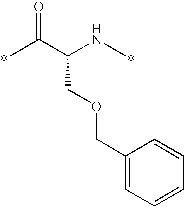 | 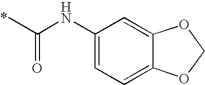 |

As will be apparent to those of skill in the art, Table I sets forth 16,000 compounds of the present invention. These compounds include, for example, compounds 1;1;1;1, 1;2;1;1, 1;3;1;1, 1;4;1;1, 1;5;1;1, 1;6;1;1, 1;7;1;1, and the like, wherein the position of the compound number reflects the specific P¹, P², P³, P⁴ residue and where the residue is located in the table. For example, the structure of compound 1;1;1;1 is as follows:

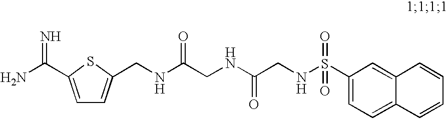

In the foregoing manner, each of the remaining 15,999 compounds of Table 1 are described to the same extent as compound 1;1;1;1 has been described.

While any combination of the elements comprising P1, P2, P3 and P4 may comprise the compounds of the present invention, certain combinations are preferred. For example, the following compounds are preferred:

| | | | | | |
|---|---|---|---|---|---|
| 1;1;1;1 | 1;1;2;2 | 1;1;3;3 | 1;1;4;4 | 1;1;5;5 | 1;1;6;6 |
| 1;1;7;7 | 1;1;8;8 | 1;1;9;9 | 1;1;10;10 | 1;1;11;11 | 1;1;12;12 |
| 1;1;13;13 | 1;1;14;14 | 1;1;15;15 | 1;1;16;16 | 1;1;17;17 | 1;1;18;18 |
| 1;1;19;19 | 1;1;20;20 | 1;1;21;21 | 1;1;22;22 | 1;1;23;23 | 1;1;24;24 |
| 1;1;25;25 | 1;1;26;26 | 1;1;27;27 | 1;1;28;28 | 1;1;29;29 | 1;1;30;30 |
| 1;1;31;31 | 1;1;32;29 | 1;1;32;32 | 1;1;33;33 | 1;1;34;34 | 1;1;35;35 |
| 1;1;36;36 | 1;1;37;37 | 1;1;38;38 | 1;1;39;39 | 1;1;40;40 | 1;2;1;1 |
| 1;2;2;2 | 1;2;3;3 | 1;2;4;4 | 1;2;5;5 | 1;2;6;6 | 1;2;7;7 |
| 1;2;8;8 | 1;2;9;9 | 1;2;10;10 | 1;2;11;11 | 1;2;12;12 | 1;2;13;13 |
| 1;2;14;14 | 1;2;15;15 | 1;2;16;16 | 1;2;17;3 | 1;2;17;17 | 1;2;18;18 |
| 1;2;19;19 | 1;2;20;20 | 1;2;21;21 | 1;2;22;22 | 1;2;23;23 | 1;2;24;24 |
| 1;2;25;25 | 1;2;26;26 | 1;2;27;27 | 1;2;28;28 | 1;2;29;29 | 1;2;30;30 |
| 1;2;31;31 | 1;2;32;29 | 1;2;32;32 | 1;2;33;33 | 1;2;34;34 | 1;2;35;35 |
| 1;2;36;36 | 1;2;37;37 | 1;2;38;38 | 1;2;39;39 | 1;2;40;40 | 1;3;1;1 |
| 1;3;2;2 | 1;3;3;3 | 1;3;4;4 | 1;3;5;5 | 1;3;6;6 | 1;3;7;7 |
| 1;3;8;8 | 1;3;9;9 | 1;3;10;10 | 1;3;11;11 | 1;3;12;12 | 1;3;13;13 |
| 1;3;14;14 | 1;3;15;15 | 1;3;16;16 | 1;3;17;17 | 1;3;18;18 | 1;3;19;19 |
| 1;3;20;20 | 1;3;21;21 | 1;3;22;22 | 1;3;23;23 | 1;3;24;24 | 1;3;25;25 |
| 1;3;26;26 | 1;3;27;27 | 1;3;28;28 | 1;3;29;29 | 1;3;30;30 | 1;3;31;31 |
| 1;3;32;32 | 1;3;33;33 | 1;3;34;34 | 1;3;35;35 | 1;3;36;36 | 1;3;37;37 |
| 1;3;38;38 | 1;3;39;39 | 1;3;40;40 | 1;4;1;1 | 1;4;2;2 | 1;4;3;3 |
| 1;4;4;4 | 1;4;5;5 | 1;4;6;6 | 1;4;7;7 | 1;4;8;8 | 1;4;9;9 |
| 1;4;10;10 | 1;4;11;11 | 1;4;12;12 | 1;4;13;13 | 1;4;14;14 | 1;4;15;15 |
| 1;4;16;16 | 1;4;17;17 | 1;4;18;18 | 1;4;19;19 | 1;4;20;20 | 1;4;21;21 |
| 1;4;22;22 | 1;4;23;23 | 1;4;24;24 | 1;4;25;25 | 1;4;26;26 | 1;4;27;27 |
| 1;4;28;28 | 1;4;29;29 | 1;4;30;30 | 1;4;31;31 | 1;4;32;12 | 1;4;32;32 |
| 1;4;33;33 | 1;4;34;34 | 1;4;35;35 | 1;4;36;36 | 1;4;37;37 | 1;4;38;38 |
| 1;4;39;39 | 1;4;40;40 | 1;5;1;1 | 1;5;2;2 | 1;5;3;3 | 1;5;4;4 |
| 1;5;5;5 | 1;5;6;6 | 1;5;7;7 | 1;5;8;8 | 1;5;9;9 | 1;5;10;10 |
| 1;5;11;11 | 1;5;12;12 | 1;5;13;13 | 1;5;14;14 | 1;5;15;15 | 1;5;16;16 |
| 1;5;17;3 | 1;5;17;17 | 1;5;18;18 | 1;5;19;19 | 1;5;20;20 | 1;5;21;21 |
| 1;5;22;22 | 1;5;23;23 | 1;5;24;24 | 1;5;25;25 | 1;5;26;26 | 1;5;27;27 |
| 1;5;28;28 | 1;5;29;29 | 1;5;30;30 | 1;5;31;31 | 1;5;32;29 | 1;5;32;32 |
| 1;5;33;33 | 1;5;34;34 | 1;5;35;35 | 1;5;36;36 | 1;5;37;37 | 1;5;38;38 |
| 1;5;39;39 | 1;5;40;2 | 1;5;40;3 | 1;5;40;40 | 1;6;1;1 | 1;6;2;2 |
| 1;6;3;3 | 1;6;4;4 | 1;6;5;5 | 1;6;6;6 | 1;6;7;7 | 1;6;8;8 |
| 1;6;9;9 | 1;6;10;10 | 1;6;11;11 | 1;6;12;12 | 1;6;13;13 | 1;6;14;14 |
| 1;6;15;15 | 1;6;16;16 | 1;6;17;17 | 1;6;18;18 | 1;6;19;19 | 1;6;20;20 |
| 1;6;21;21 | 1;6;22;22 | 1;6;23;23 | 1;6;24;24 | 1;6;25;25 | 1;6;26;26 |
| 1;6;27;27 | 1;6;28;28 | 1;6;29;29 | 1;6;30;30 | 1;6;31;31 | 1;6;32;32 |
| 1;6;33;33 | 1;6;34;34 | 1;6;35;35 | 1;6;36;36 | 1;6;37;37 | 1;6;38;38 |
| 1;6;39;39 | 1;6;40;40 | 1;7;1;1 | 1;7;2;2 | 1;7;3;3 | 1;7;4;4 |
| 1;7;5;5 | 1;7;6;6 | 1;7;7;7 | 1;7;8;8 | 1;7;9;9 | 1;7;10;10 |
| 1;7;11;11 | 1;7;12;12 | 1;7;13;13 | 1;7;14;14 | 1;7;15;6 | 1;7;15;8 |
| 1;7;15;15 | 1;7;16;16 | 1;7;17;17 | 1;7;18;18 | 1;7;19;19 | 1;7;20;20 |
| 1;7;21;21 | 1;7;22;6 | 1;7;22;8 | 1;7;22;22 | 1;7;23;23 | 1;7;24;24 |
| 1;7;25;25 | 1;7;26;26 | 1;7;27;27 | 1;7;28;28 | 1;7;29;29 | 1;7;30;30 |
| 1;7;31;31 | 1;7;32;12 | 1;7;32;32 | 1;7;33;33 | 1;7;34;34 | 1;7;35;35 |
| 1;7;36;36 | 1;7;37;37 | 1;7;38;38 | 1;7;39;39 | 1;7;40;40 | 1;8;1;1 |
| 1;8;2;2 | 1;8;3;3 | 1;8;4;4 | 1;8;5;5 | 1;8;6;6 | 1;8;7;7 |
| 1;8;8;8 | 1;8;9;9 | 1;8;10;10 | 1;8;11;11 | 1;8;12;12 | 1;8;13;13 |
| 1;8;14;14 | 1;8;15;15 | 1;8;16;16 | 1;8;17;3 | 1;8;17;17 | 1;8;18;18 |
| 1;8;19;19 | 1;8;20;20 | 1;8;21;21 | 1;8;22;22 | 1;8;23;23 | 1;8;24;24 |
| 1;8;25;25 | 1;8;26;26 | 1;8;27;27 | 1;8;28;28 | 1;8;29;29 | 1;8;30;30 |
| 1;8;31;31 | 1;8;32;29 | 1;8;32;32 | 1;8;33;33 | 1;8;34;34 | 1;8;35;35 |
| 1;8;36;36 | 1;8;37;37 | 1;8;38;38 | 1;8;39;39 | 1;8;40;40 | 1;9;1;1 |
| 1;9;2;2 | 1;9;3;3 | 1;9;4;4 | 1;9;5;5 | 1;9;6;6 | 1;9;7;7 |
| 1;9;8;8 | 1;9;9;9 | 1;9;10;10 | 1;9;11;11 | 1;9;12;12 | 1;9;13;13 |
| 1;9;14;14 | 1;9;15;15 | 1;9;16;16 | 1;9;17;17 | 1;9;18;18 | 1;9;19;19 |
| 1;9;20;20 | 1;9;21;21 | 1;9;22;22 | 1;9;23;23 | 1;9;24;24 | 1;9;25;25 |
| 1;9;26;26 | 1;9;27;27 | 1;9;28;28 | 1;9;29;29 | 1;9;30;30 | 1;9;31;31 |
| 1;9;32;32 | 1;9;33;33 | 1;9;34;34 | 1;9;35;35 | 1;9;36;36 | 1;9;37;37 |
| 1;9;38;38 | 1;9;39;39 | 1;9;40;40 | 1;10;1;1 | 1;10;2;2 | 1;10;3;3 |
| 1;10;4;4 | 1;10;5;5 | 1;10;6;6 | 1;10;7;7 | 1;10;8;8 | 1;10;9;9 |
| 1;10;10;10 | 1;10;11;11 | 1;10;12;12 | 1;10;13;13 | 1;10;14;14 | 1;10;15;15 |
| 1;10;16;16 | 1;10;17;3 | 1;10;17;17 | 1;10;18;18 | 1;10;19;19 | 1;10;20;20 |
| 1;10;21;21 | 1;10;22;8 | 1;10;22;22 | 1;10;23;23 | 1;10;24;24 | 1;10;25;25 |
| 1;10;26;26 | 1;10;27;27 | 1;10;28;28 | 1;10;29;29 | 1;10;30;30 | 1;10;31;31 |
| 1;10;32;12 | 1;10;32;32 | 1;10;32;34 | 1;10;33;12 | 1;10;33;33 | 1;10;34;34 |
| 1;10;35;35 | 1;10;36;36 | 1;10;37;37 | 1;10;38;38 | 1;10;39;34 | 1;10;39;39 |
| 1;10;40;3 | 1;10;40;8 | 1;10;40;40 | | | |

A further aspect of the present invention is determining the structure of any compound identified as a modulator. It is within the scope of the present invention that chemical structures of compounds identified as having a desired characteristic can be determined by deconvolution of the library (see, Smith et al., Bio. Med. Chem. Lett. 4, 2821 (1994); Kurth et al., J. Org. Chem. 59, 5862 (1994); Murphy et al, J. Am. Chem. Soc. 117, 7029 (1995); Campell et al., J. Am. Chem. Soc. 118, 5381 (1995); and Erb et al., Proc. Natl. Acad. Sci. USA 91, 11422 (1994)). In addition, deconvolution procedures can be verified by analysis of the cleaved compound, such as by mass spectrometry.

The libraries of the present invention are useful as screening tools for discovering new lead structures by evaluation across an array of biological assays, including the discovery of selective inhibition patterns across isozymes. A library can be tested with the ligands attached to the solid supports as depicted in Formula I, or the compounds can be detached prior to evaluation (Formula Ia). With the compounds of Formula I, screening assays such as FACS sorting and cell lawn assays can be used.

In another aspect, the present invention provides a method for modulating a protease comprising: contacting the protease with a compound having formula I, thereby modulating the protease.

Various assays can be used in the present invention. These assays include, but are not limited to, an endotheliase catalytic assay, a matripase catalytic assay and a urokinase catalytic assay. Suitable assays are set forth in Example 3.

In a lawn assay, a library of solid supports, preferably beads, is screened for the ability of compounds on the supports to affect the activity of an enzyme. Using the lawn assay, supports containing the active compounds are quickly and easily located merely by viewing zones of inhibition in a matrix. In one embodiment, the solid supports are contacted with a colloidal matrix such as agarose. The compounds are linked to the supports by a cleavable linker and released, e.g., by exposure to light. As they slowly diffuse out of the solid supports, zones of high concentration of the compounds are created in the supports' immediate vicinity. The compounds contact enzyme contained in the matrix. Substrate is contacted with the matrix and reacts with the enzyme. Conversion of substrate to product is measured by monitoring a photometric change in the substrate or in a coenzyme or cofactor involved in reaction. For example, the substrate can be fluorogenic, i.e., becoming fluorescent when converted to product. In this case, compounds that are active inhibitors of the enzyme reaction are detected as dark zones of inhibition. The less active, or inactive, compounds are contained in the lighter areas.

Using this assay, positive results from an assay of a combinatorial library can be detected very quickly. Furthermore, compound activity can be quantitated by, for example, comparing the sizes of zones of activity. Once zones of activity have been determined, the relevant supports at the center of the zones can be located and the active compounds on those supports identified. The lawn assay thus allows large libraries of compounds to be quickly and easily screened. Very little effort is required to array the solid supports or assay the compounds released from the supports.

Enzymes that can be used in the assay include, but are not limited to, acid phosphatase, activated Protein C, alkaline phosphatase, aminopeptidases B & M, amyloid A4-generating enzyme, angiotensinase, aryl sulfatase, β-galactosidase, β-glucosidase, β-glucuronidase, calpains I & II, cathepsins B, C, D & G, cholinesterase, chymotrypsin, collagenase, dipeptidyl peptidases I-IV, elastase, endothelin converting enzyme, Factor Xa, Factor XIa, granzymes A & B, HIV protease, IL- I B Convertase, kallikrein, lysozyme, mast cell protease, peroxidase, plasmepsin I and II, plasmin, prohormone convertase, renin, serine proteases (e.g., Type II membrane bound serine proteases, such as matriptase), spleen fibrinolytic proteinase, staphylocoagulase, thrombin, tissue plasminogen activator, trypsin, tryptase urokinase, and xanthine oxidase. Those of skill in the art will know of other enzyme systems suitable for modulation with the present compounds.

Having generally described the invention with respect to the preferred embodiments thereof, the following specific exemplary disclosure is provided. Unless otherwise noted, all materials employed in the following examples are readily available from commercial sources.

### EXAMPLES

### Example 1

In this example, a library of 16,000 compounds is prepared according to methods of the present invention.

| | |
|---|---|
| 1 | P¹ group, amidinothiophene, as the attachment point to the resin. |
| 10 | P² groups (Gly, Ala, Leu, Phe, Arg, Glu, Ser, Pro, Gin, and hydroxy- |
| | Pro, all L). |
| 40 | P³ groups (the L and D versions of the amino acids at P² + 21 other |
| | natural and unnatural amino acids). |
| 40 | P⁴ groups, subdivided into 10 each of sulfonamides, amides, |
| | carbamates, and ureas. |
| 1 | 1 × 10 × 40 × 40 = 16,000 total distinct compounds. |

The different reaction conditions for each different class of P⁴ groups is one way to organize the library. In order to explore the differences among P² groups, a “P² Library” of 40 mixtures is prepared.

| | | | | | | | | | | |
|---|---|---|---|---|---|---|---|---|---|---|
| P2-Library | P²a | P²b | P²c | P²d | P²e | P²f | P²g | P²h | P²i | P²j |

| | | |
|---|---|---|
| P⁴ sulfon- | a-X- | |
| amides-4s | 4s | |
| P⁴ amides-4a | | |
| P⁴ carba- | | a-X- |
| mates-4c | | 4s |
| P⁴ ureas-4u | | |

Each of the 40 mixtures is composed of a single P² (e.g., a), the mixture of all 40 P³'s (X), and a 10-member subset of the 40 P⁴'s (e.g., 4s). Thus, each mixture contains 1×40×10=400 members, and each of the 16,000 library compounds is found in exactly one mixture. Since each mixture would only contain a single P², this library provides clear evidence for which P²'s provides active compounds in combination with any of the P³'s and P⁴'s, and even narrows down active P⁴ groups by linker type.

In order to get the same information for the other two points of diversity, the same 16,000 compounds are synthesized twice more, varying each of the other two positions in a similar fashion. The 40 P³'s are split among 4 sublibraries to maintain the same 4×10 mixture format, which eases screening and data management. One of these libraries is depicted below:

| | P³ | P³ | P³ | P³ | P³ | P³ | P³ | P³ | P³ | P³ |
|---|---|---|---|---|---|---|---|---|---|---|
| P³-Library 2 | 11 | 12 | 13 | 14 | 15 | 16 | 17 | 18 | 19 | 20 |

| | | |
|---|---|---|
| P⁴ sulfonamides-4s | a-X- | |
| | 4s | |
| P⁴ amides-4a | | |
| P⁴ carbamates-4c | | a-X- |
| | | 4c |
| P⁴ ureas-4u | | |

Each of the 40 mixture is composed of all 10 P²'s (X), a single P³ (e.g., 11), and a 10-member subset of the 40 P⁴'s (e.g., 4s). Thus, each mixture contains 1×10×10=100 members, and the four sub-libraries together contain all 16,000 compounds (see Table I).

Altogether, 240 mixtures of either 100 or 400 members each, in six 40-member libraries are produced, with 16,000 individual compounds each represented three times. Direct information about each of the 3 points of diversity, P², P³, and P⁴ cap, is obtained.

### Example 2

In this example, various solid supports are utilized according to methods of the present invention.

Upon loading of the P¹ amidine on the Wang carbonate resin, the amidine is protected as a carbamate, eliminating the possibility of further reaction at the second nitrogen center. Furthermore, the linkage is considerably more robust than the tritylamine linkage, requiring concentrated TFA for cleavage. This opens up the possibility for selective cleavage of acid-labile protecting groups used during the synthesis prior to cleavage of the library from the resin, preventing the protecting group fragments from becoming part of the final mixtures.

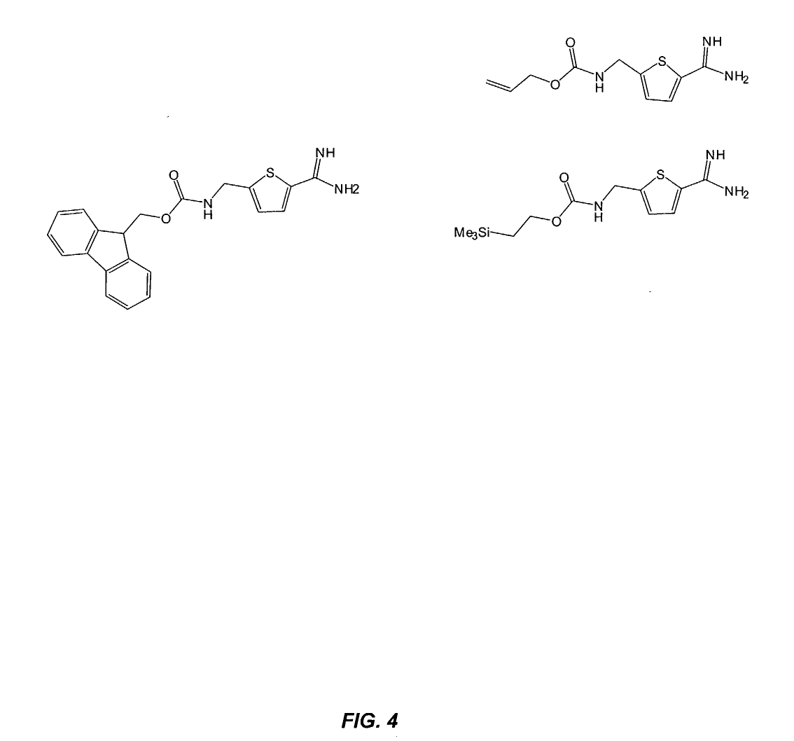

One material used for resin loading studies was 2-(Fmoc-aminomethyl)-5-amidinothiophene. In initial experiments, some deprotection of the Fmoc was seen during the loading conditions, so a variety of other protecting groups have been considered, including the Alloc-protected version as well as the unsubstituted methyl amidinothiophene. In addition, the Teoc compound has also been prepared. The following protecting groups have been used: 1) Alloc, 2) Teoc, and 3) Fmoc (see, FIG. 4).

Many different sets of loading conditions and control experiments have been made with the resin and loading reaction milieu in the absence of amidine. Results show loading efficiencies in the range of 60-70% with the methyl test compound, Fmoc, and Alloc under the identified conditions, although some small amount of Fmoc deprotection is noted. Solution-phase test reactions support these data. It appears that when DBU is used as the base in the loading reaction that there is competition of some sort for addition to the resin, as evidenced by the theoretically-complete liberation of p-nitrophenol upon the addition of DBU to the resin in the absence of a reactive P¹ fragment.

Since initial results in evaluating the resin loading were successful, a concurrent effort was made to validate the solid phase synthesis of a typical protease inhibitor. The two batches resulted from parallel reactions in THF and DMF as solvent. The DMF batch produced a much better yield (69% vs. 37% overall), and the final product was ˜96% pure.

### Example 3

In this example, various enzymes are used to assess compound activity.

Combinatorial Library Screening

Each library vial contained between 6 to 22 mgs of either one hundred or four hundred individual lyophilized compounds, and was reconstituted in DMSO to a similar stock concentration (˜5.0 mM), and allowed to equilibrate for 30 min at ambient temperature. This stock solution was then diluted into assay buffer, and allowed to equilibrate for 30 min at ambient temperature. The assay buffer was HBS (10 mM HEPES, 150 mM sodium chloride pH 7.4) with 0.1% BSA.

Initial screen determinations were conducted by combining in appropriate wells of a Coming microtiter plate, 25 microliters of the test compound diluted in assay buffer (or buffer alone for Vo (uninhibited velocity) measurement), 50 microliters of the substrate and 50 microliters of the enzyme diluted in buffer, yielding a final volume of 125 microliters. The initial velocity of substrate hydrolysis was measured by the change of absorbance at 405 nM using a Thermo Max® Kinetic Microplate Reader over a 5 minute period at ambient temperature in which less than 5% of the added substrate was utilized. Results were reported as the percent inhibition of Vo (uninhibited velocity) by the inhibitors.

| | | | | Individual |
|---|---|---|---|---|
| | | | | Inhibitor |
| Enzyme | [final] | Substrate | [final] | [final] |
| Endotheliase | 250 pM | Methylsulfonyl-D- | 600 uM | 50 nM |
| | | cyclohexyltyrosyl- | | |
| | | glycyl-arginine-p- | | |
| | | nitroaniline acetate | | |
| Matriptase | 250 pM | N-α-Benzyloxycarbonyl- | 200 uM | 50 nM |
| | | D-arginyl-L-glycyl-L- | | |
| | | arginine-p-nitroaniline | | |
| | | dihydrochloride | | |
| uPA | 1.8 nM | L-Pyroglutamyl-glycyl- | 300 uM | 10 nM |
| | | L-arginine-p- | | |
| | | nitroaniline hydrochloride | | |

Urokinase Catalytic Assay

Urokinase catalytic activity was determined using the chromogenic substrate S-2444 (L-Pyroglutamyl-glycyl-L-arginine-p-nitroaniline hydrochloride), obtained from DiaPharma Group, Inc. Urokinase (Abbokinase), manufactured by Abbott Laboratories, was obtained from Priority Pharmaceuticals.

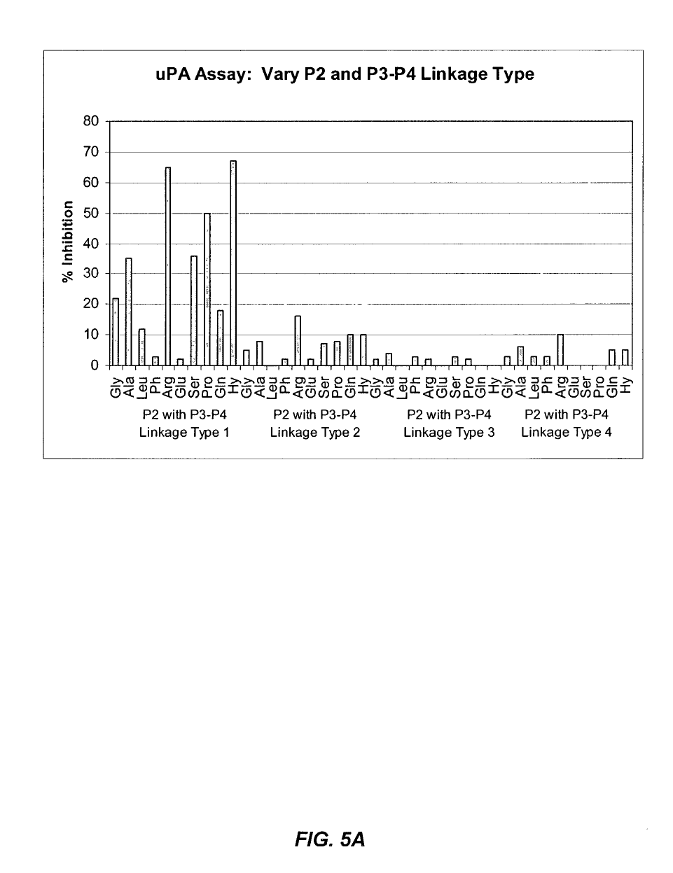

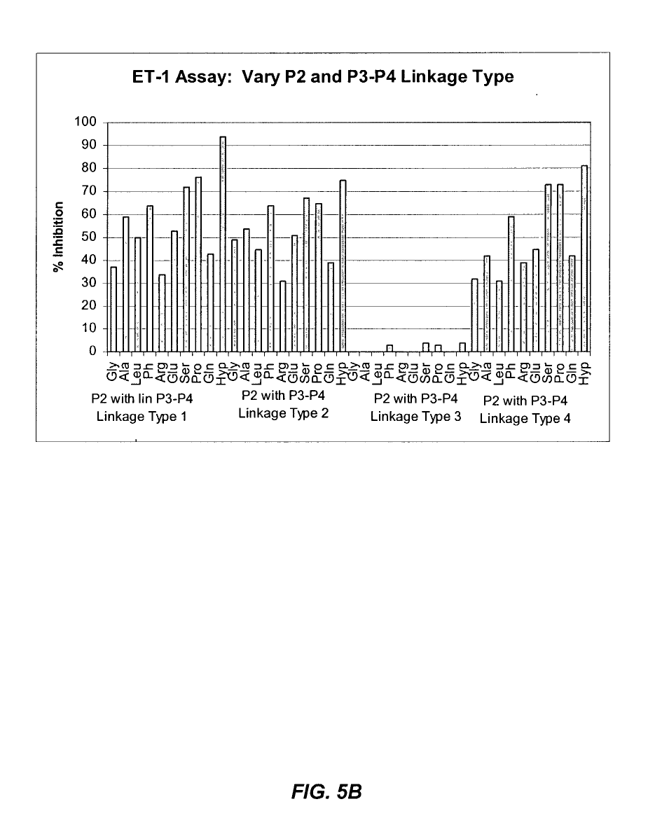

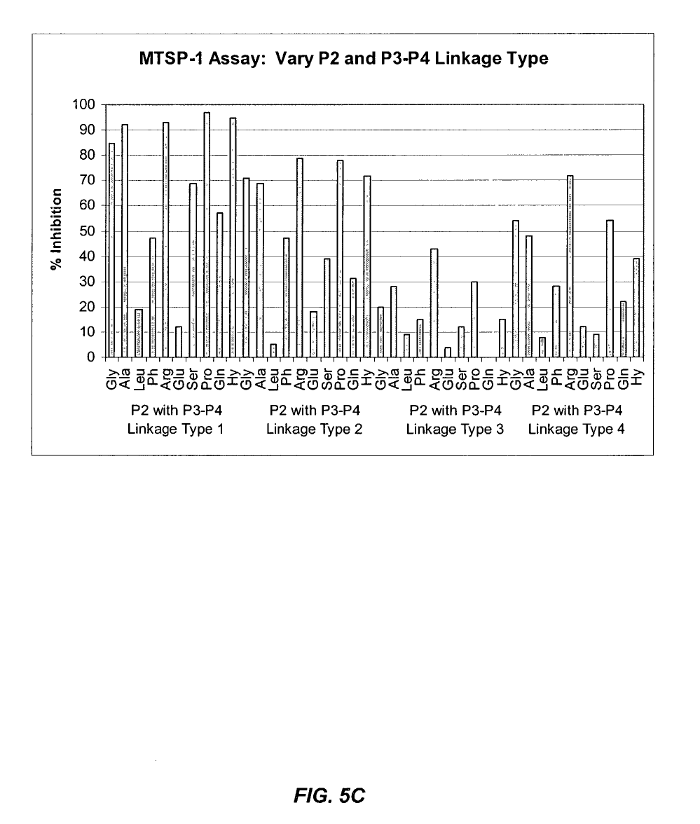

FIG. 5A is a histogram plot which illustrates the degree of inhibition for the urokinase enzyme while varying the P2 segment and the P3-P4 linkage type of a library of the present invention. In this library, which corresponds to Table 1, there are 10 possible P2's, and 4 possible P3-P4 linkage types. As shown in FIG. 5A, linkage Type 1 is the most active.

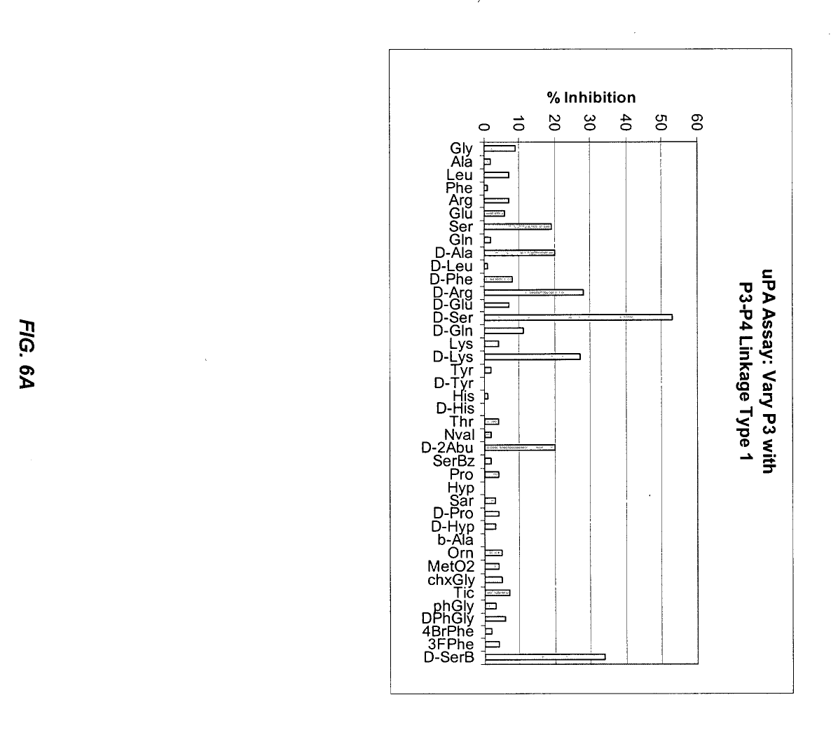

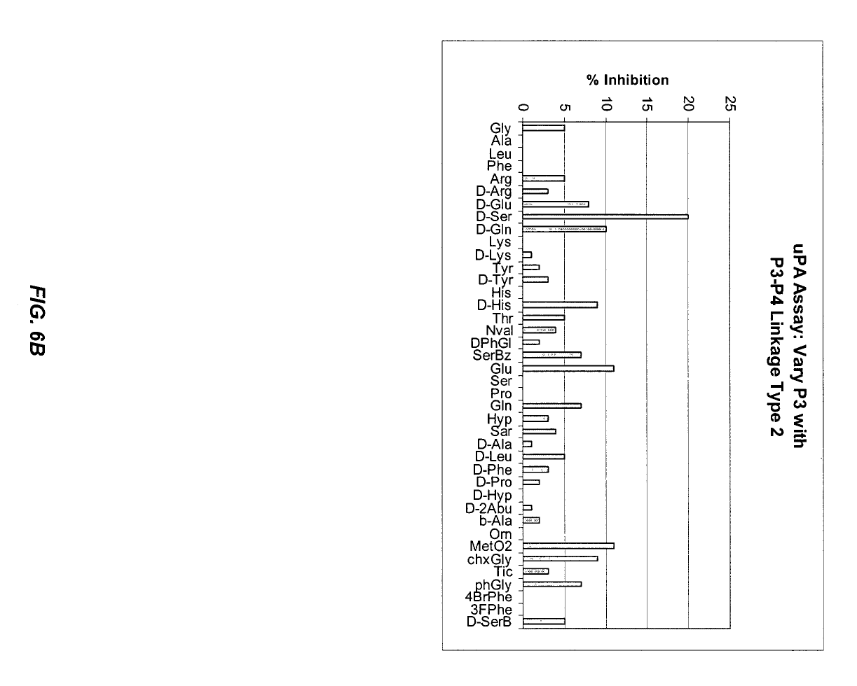

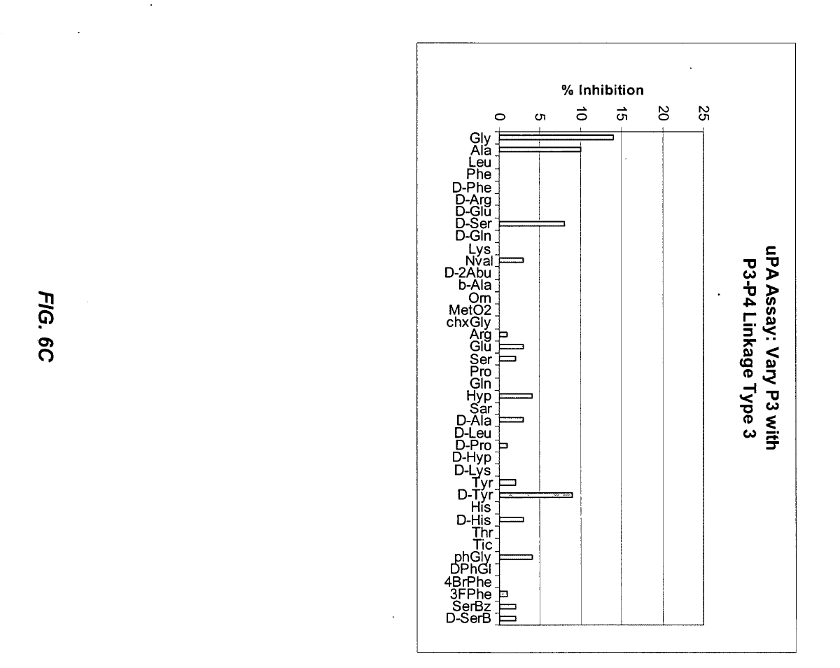

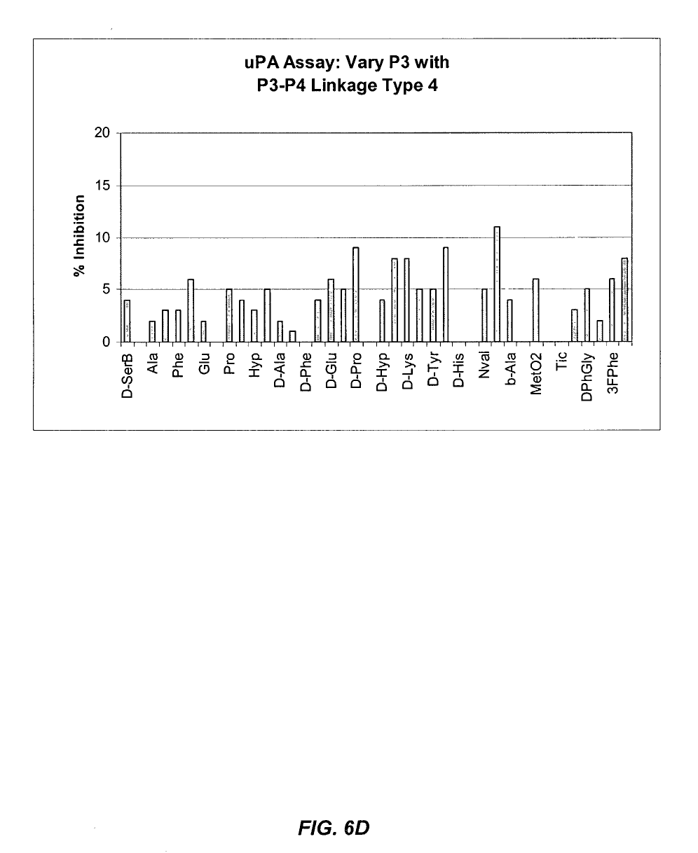

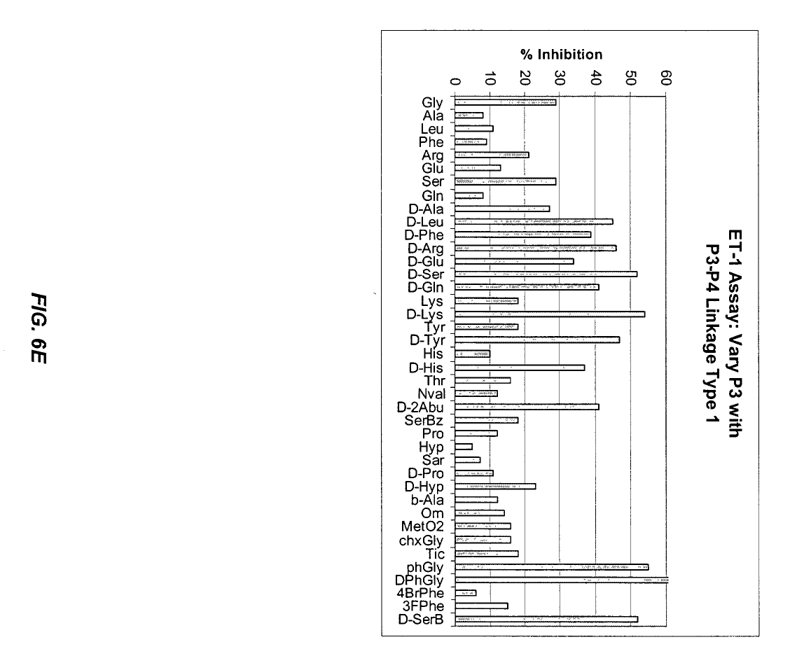

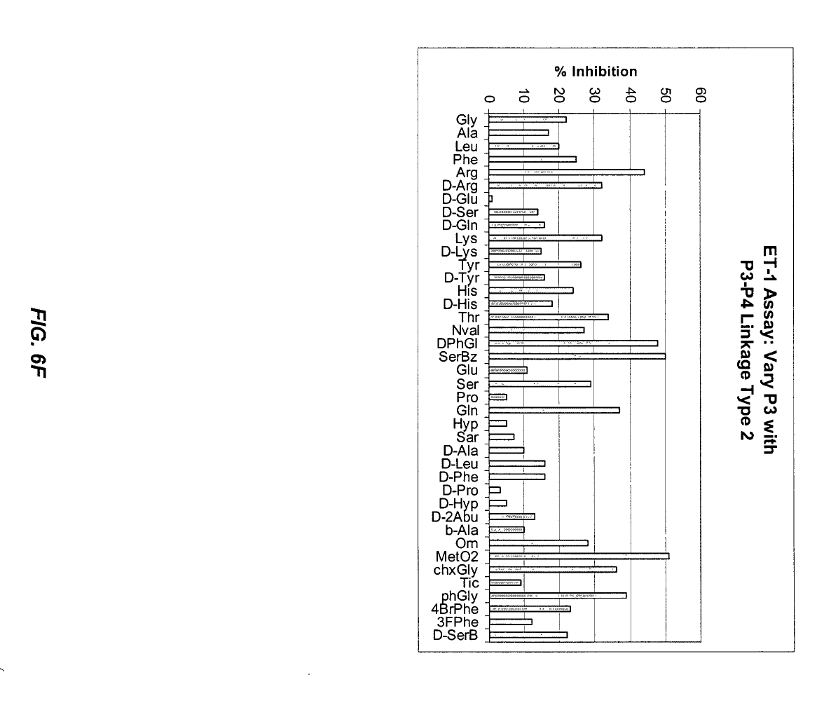

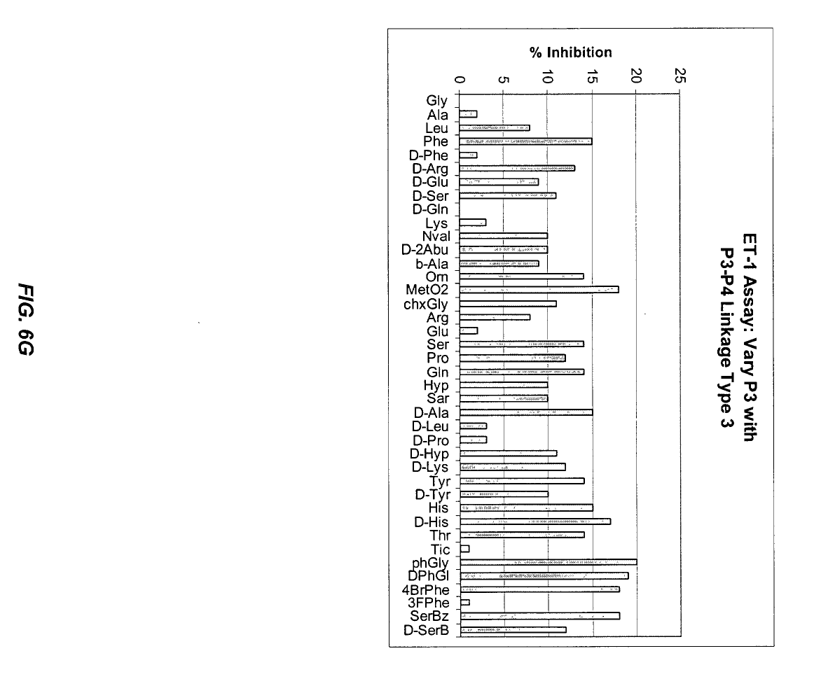

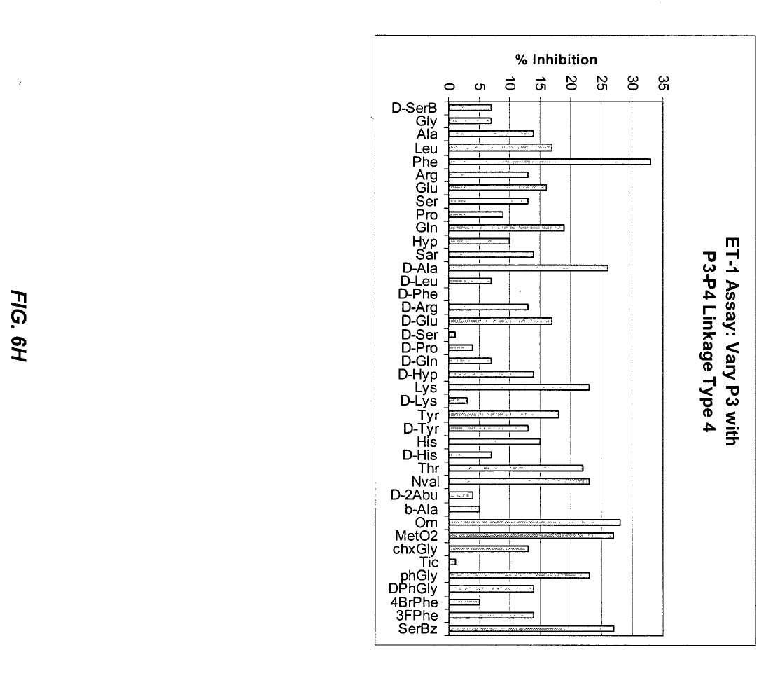

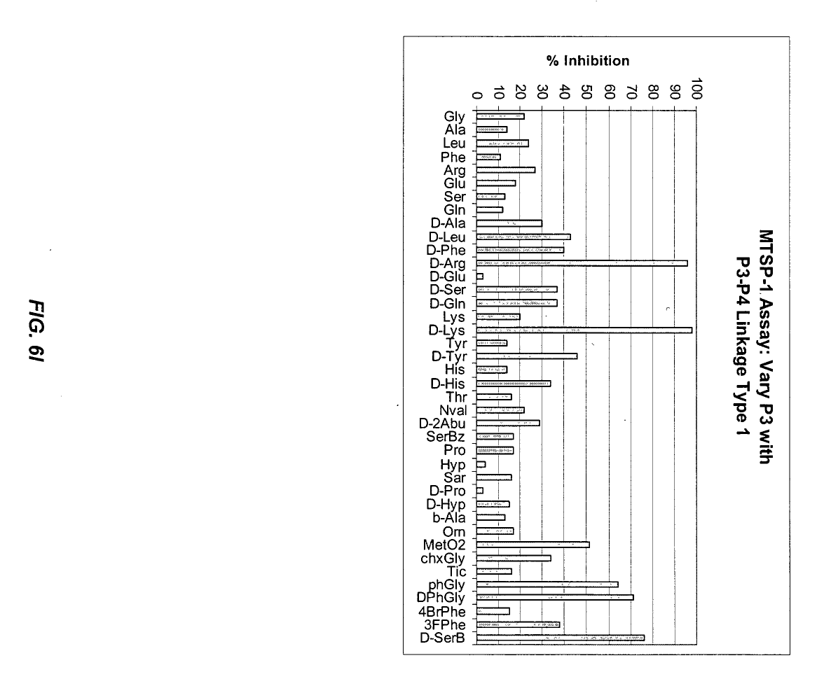

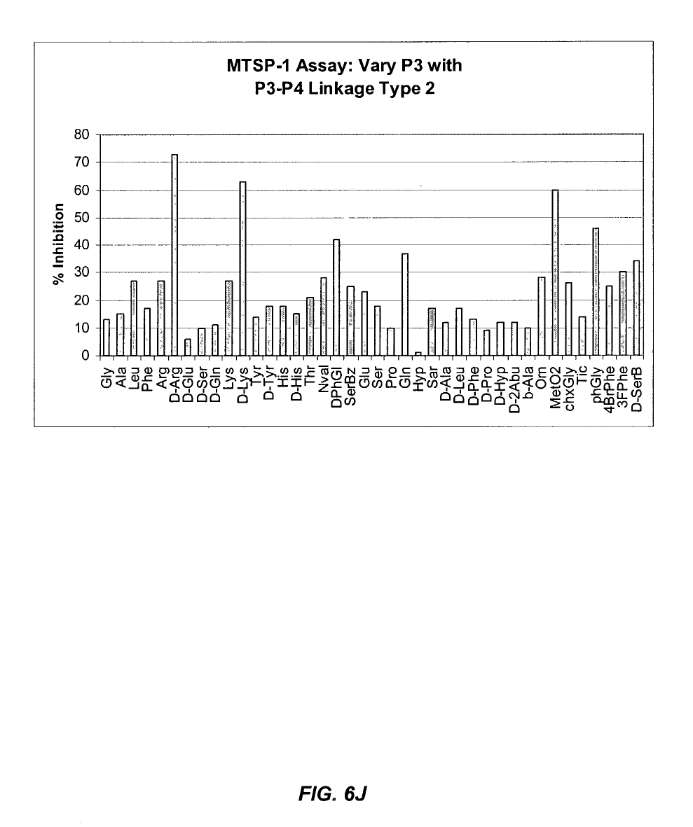

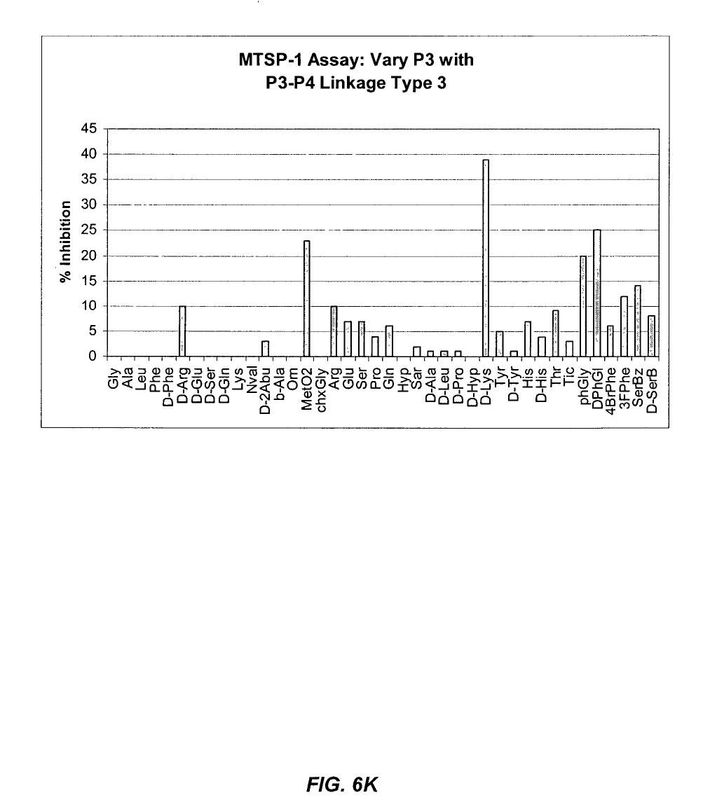

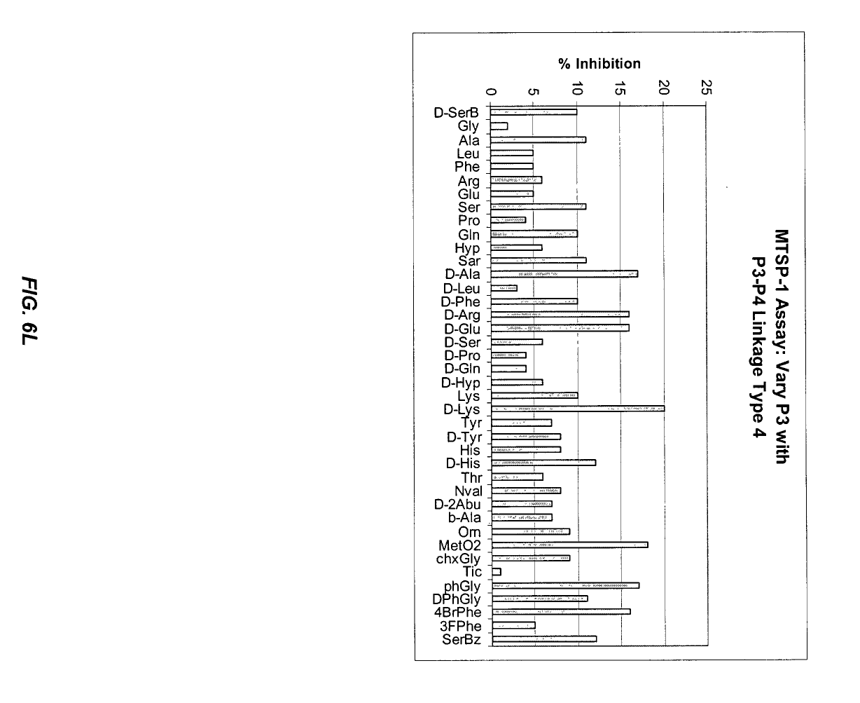

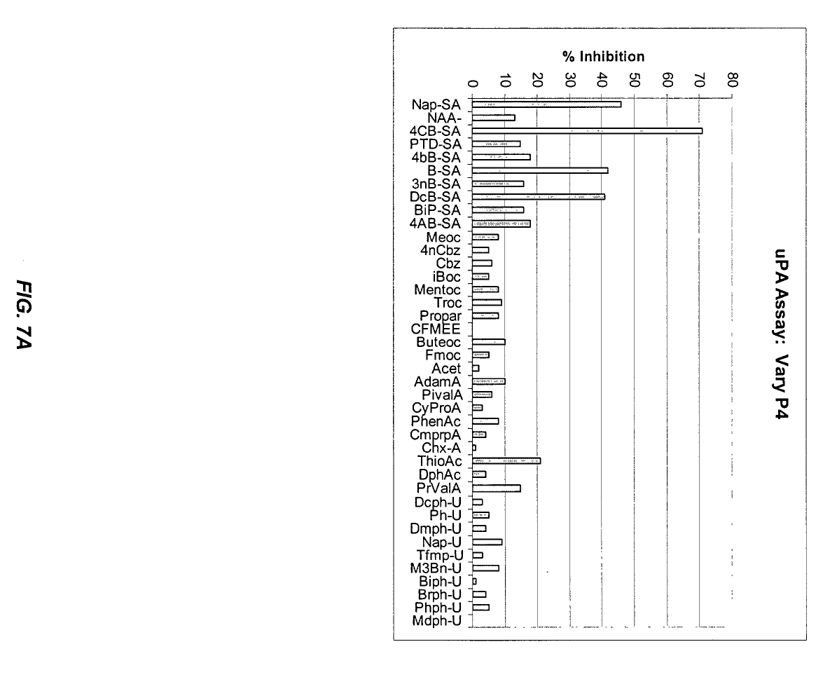

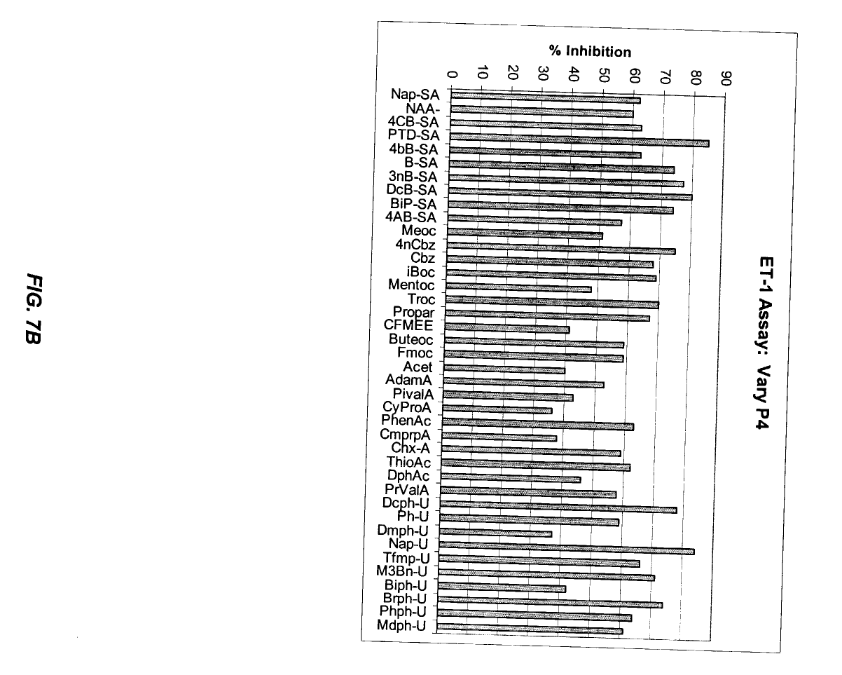

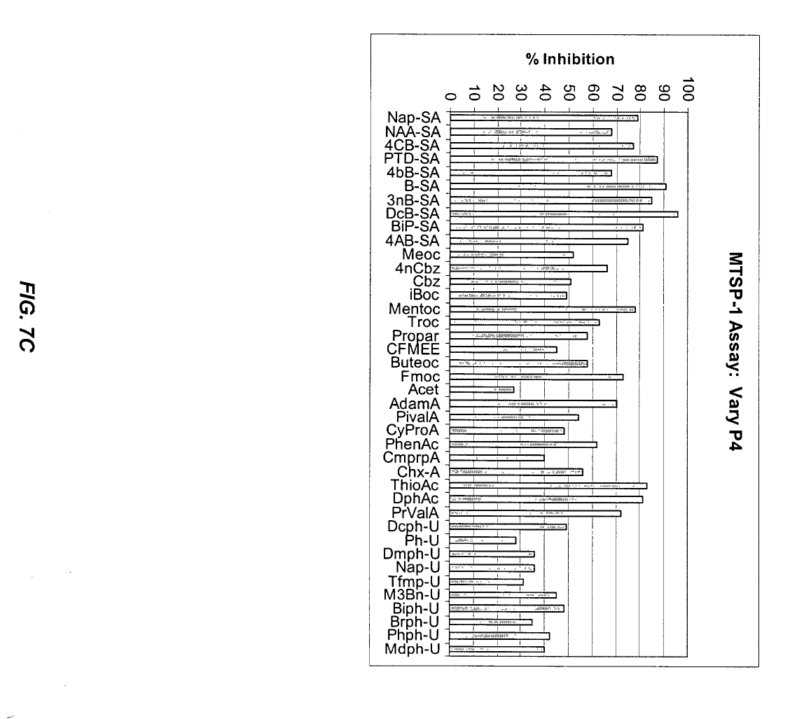

FIGS. 6A-D illustrate the degree of inhibition for the urokinase enzyme while varying the P3 segment and having 4 different P3-P4 linkages. With reference to Table 1, in this library there are 40 possible P3's as shown on the x-axis. FIGS. 6A-D shows the various P3-P4 linkage types. FIG. 7A illustrates the degree of inhibition for the urokinase enzyme while varying the P4 segment. As shown in Table 1, in this particular library there are 40 different P4's as shown on the x-axis of FIG. 7A. In this study, 4CB-SA was the most active.

Endotheliase Catalytic Assay

Endotheliase catalytic activity was determined using the chromogenic substrate Spectrozyme tPA (Methylsulfonyl-D-cyclohexyltyrosyl-glycyl-arginine paranitroaniline acetate), obtained from American Diagnostica, Inc. Recombinant Endotheliase was manufactured by Corvas, International.

FIG. 5B is a histogram plot which illustrates the degree of inhibition for the endotheliase enzyme while varying the P2 segment and the P3-P4 linkage type of a library of the present invention. In this library, which corresponds to Table 1, there are 10 possible P2's, and 4 possible P3-P4 linkage types. As shown in FIG. 5B, linkage Type 3 is the least active.

FIGS. 6E-H illustrate the degree of inhibition for the endotheliase enzyme while varying the P3 segment and having 4 different P3-P4 linkages. With reference to Table 1, in this library there are 40 possible P3's as shown on the x-axis. FIGS. 6E-H shows the various P3-P4 linkage types.

FIG. 7B illustrates the degree of inhibition for the endotheliase enzyme while varying the P4 segment. As shown in Table 1, in this particular library there are 40 different P4's as shown on the x-axis of FIG. 7B. In this study, PTD-SA was the most active.

Matriptase Catalytic Assay

Matriptase catalytic activity was determined using the chromogenic substrate S-2765 (N-α-Benzyloxycarbonyl-D-arginyl-L-glycyl-L-arginine-p-nitroaniline dihydrochloride), obtained from DiaPharma Group, Inc. Recombinant Matriptase was manufactured by Corvas, International.

FIG. 5C is a histogram plot which illustrates the degree of inhibition for the matriptase enzyme while varying the P2 segment and the P3-P4 linkage type of a library of the present invention. In this library, which corresponds to Table 1, there are 10 possible P2's, and 4 possible P3-P4 linkage types. As shown in FIG. 5, linkage Type 1 is the most active.

FIGS. 6I-L illustrate the degree of inhibition for the matriptase enzyme while varying the P3 segment and having 4 different P3-P4 linkages. With reference to Table 1, in this library there are 40 possible P3's as shown on the x-axis. FIGS. 6I-L shows the various P3-P4 linkage types.

FIG. 7C illustrates the degree of inhibition for the matriptase enzyme while varying the P4 segment. As shown in Table 1, in this particular library there are 40 different P4's as shown on the x-axis of FIG. 7C. In this study, DcB-SA was the most active.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A combinatorial library comprising compounds of the formula:
●-P¹—(P²)_{y}—P⁴ I
wherein:
P¹ is an arginine surrogate attached directly to a solid support;
P² is an amino acid residue;
y is an integer from 1 to about 12 wherein each P² can be the same or different; and
P⁴ is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group.

2. The combinatorial library of claim 1, wherein 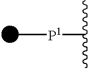
is said arginine surrogate attached directly to said solid support having the formula: 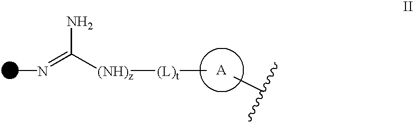
wherein the ring ‘A’ is a member selected from the group consisting of a five- or a six-membered optionally substituted cycloalkyl group, optionally substituted aryl group and an optionally substituted heteroaryl group, wherein said heteroaryl group has from 1 to 3 heteroatoms selected from the group consisting of optionally substituted N, O, and S; and
z is 0 or 1;
L is selected from the group consisting of alkylene optionally interrupted by a heteroatom and S; and
t is 0 or 1.

3. The combinatorial library of claim 2, wherein 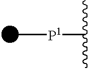
is said arginine surrogate attached directly to said solid support having the formula: 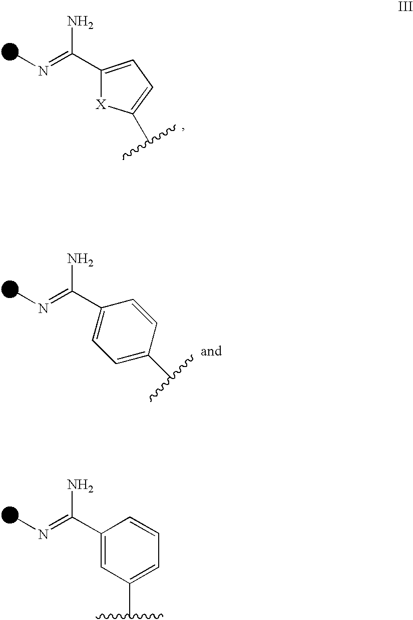
wherein X is a heteroatom selected from the group consisting of optionally substituted N, S and O.

4. The combinatorial library of claim 1, wherein each P² is an amino acid residue independently selected from the group consisting of an L-amino acid, a D-amino acid, sarcosine (Sar), norvaline (Nval), β-alanine (β-Ala), methionine sulfone (Met(O₂), cyclohexyl-glycine (chx-Gly), tetrahydro-3-isoquinolinecarboxylic acid (Tic), phenylglycine (phGly), 4-bromophenylalanine (4BrPhe), 3-fluorophenylalanine (3FPhe), and D-O-benzylserine Ser(Bz).

5. The combinatorial library of claim 2, wherein 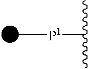
has the formula 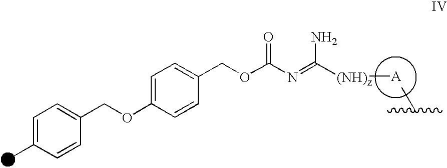
wherein the ring ‘A’ is a member selected from the group consisting of a five- or a six-membered optionally substituted cycloalkyl group, optionally substituted aryl group and an optionally substituted heteroaryl group, wherein said heteroaryl group has from 1 to 3 heteroatoms selected from the group consisting of optionally substituted N, O, and S.

6. The combinatorial library of claim 5, wherein said solid support is derived from a member selected from the group consisting of 4-nitrophenyl carbonate resin, imidazole carbonate resin and succinimidyl carbonate resin.

7. The combinatorial library of claim 5, wherein 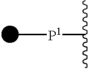
has the formula 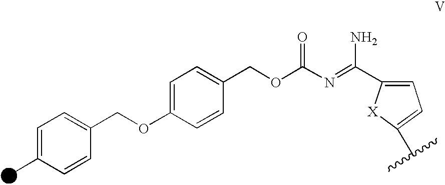
wherein:
X is a heteroatom selected from the group consisting of S, O and optionally substituted N.

8. The combinatorial library of claim 3, wherein said compounds have the formula: 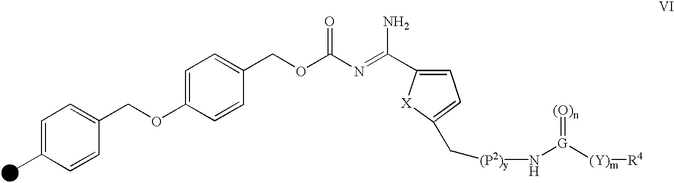
wherein:
X is a heteroatom selected from the group consisting of optionally substituted N, S and O;
each P² may be the same or a different amino acid residue;
y is an integer of 1 to about 3;
G is carbon and n is 1; or alternatively,
G is sulfur and n is 0, 1 or 2;
Y is a heteroatom selected from the group consisting of optionally substituted N and O;
m is 0 or 1; and
R⁴ is an optionally substituted aryl group, an optionally substituted heteroaryl group, an optionally substituted arylalkyl group, an optionally substituted heteroarylalkyl group and an optionally substituted alkyl group.

9. A combinatorial library comprising compounds of the formula:
P¹—(P²)_{y}—P⁴ Ia
wherein:
P¹ is an arginine surrogate;
P² is an amino acid residue;
y is an integer from 1 to about 12 wherein each P² can be the same or different; and
P⁴ is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group.

10. The combinatorial library of claim 9, wherein the compounds have the formula 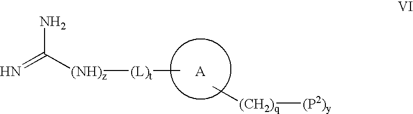
wherein the ring ‘A’ is a member selected from the group consisting of a five- or a six-membered optionally substituted cycloalkyl group, optionally substituted aryl group and an optionally substituted heteroaryl group, wherein said heteroaryl group has from 1 to 3 heteroatoms selected from the group consisting of optionally substituted N, O, and S; and
z is 0 or 1;
L is selected from the group consisting of alkylene optionally interrupted by a heteroatom and S;
t is 0 or 1;
q is an integer from 0 to about 3; and
y is an integer from 1 to about 12 wherein each P² can be the same or different.

11. The combinatorial library of claim 9, wherein the compounds have the formula 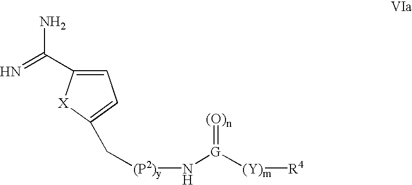
wherein:
X is a heteroatom selected from the group consisting of N, S and O;
each P² may be the same or a different amino acid residue;
y is an integer of 1 to about 3;
G is carbon and n is 1; or alternatively,
G is sulfur and n is 0, 1 or 2;
Y is a heteroatom selected from the group consisting of optionally substituted N and O;
m is 0 or 1; and
R⁴ is an optionally substituted aryl group, an optionally substituted heteroaryl group, an optionally substituted arylalkyl group, an optionally substituted heteroarylalkyl group or an optionally substituted alkyl group.

12. The combinatorial library of claim 9, wherein each P² is an amino acid residue independently selected from the group consisting of an L-amino acid, a D-amino acid, sarcosine (Sar), norvaline (Nval), β-alanine (β-Ala), methionine sulfone (Met(O₂), cyclohexyl-glycine (chx-Gly), tetrahydro-3-isoquinolinecarboxylic acid (Tic), phenylglycine (phGly), 4-bromophenylalanine (4BrPhe), 3-fluorophenylalanine (3FPhe), and D-O-benzylserine Ser(Bz).

13. A method for preparing a protease modulator having an arginine surrogate on a solid support, said method comprising:
(a) attaching a protected arginine surrogate to said solid support to provide a protected support-bound arginine surrogate;
(b) deprotecting said support-bound arginine surrogate to form a deprotected support-bound arginine surrogate;
(c) contacting said deprotected support-bound arginine surrogate with at least one protected amino acid under conditions to provide a protected support-bound substituted arginine surrogate;
(d) deprotecting said protected support-bound substituted arginine surrogate to form a deprotected substituted support-bound arginine surrogate; and
(e) contacting said deprotected support-bound substituted arginine surrogate with at least one capping agent under conditions to provide a protease modulator having an arginine surrogate on said solid support.

14. The method for preparing a protease modulator of claim 13, further comprising repeating step (c) and step (d) in iterative fashion with about 2 to about 12 protected amino acids.

15. The method for preparing a protease modulator of claim 13, further comprising:
(f) removing said protease modulator having an arginine surrogate from said solid support.

16. The method for preparing a protease modulator of claim 13, wherein said protected arginine surrogate has the formula: 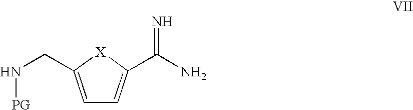
wherein:
X is a heteroatom selected from the group consisting of N, S and O; and
PG is a protecting group.

17. The method for preparing a protease modulator of claim 16, wherein said PG is a member selected from the group consisting of Fmoc, Alloc and Teoc.

18. The method for preparing a protease modulator of claim 13, wherein said protected amino acid is an Fmoc protected amino acid.

19. The method for preparing a protease modulator of claim 18, wherein the amino acid of said Fmoc protected amino acid is a member selected from the group consisting of an L-amino acid, a D-amino acid, sarcosine (Sar), norvaline (Nval), β-alanine (β-Ala), methionine sulfone (Met(O₂), cyclohexyl-glycine (chx-Gly), tetrahydro-3-isoquinolinecarboxylic acid (Tic), phenylglycine (phGly), 4-bromophenylalanine (4BrPhe), 3-fluorophenylalanine (3FPhe), and D-O-benzylserine Ser(Bz).

20. The method for preparing a protease modulator of claim 13, wherein said capping agent is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group and an optionally substituted alkyl group.

21. The method for preparing a protease modulator of claim 13, wherein said solid support is derived from a member selected from the group consisting of 4-nitrophenyl carbonate resin, imidazole carbonate resin and succinimidyl carbonate resin.

22. The method for preparing a protease modulator of claim 13, wherein said protease modulator having an arginine surrogate on said solid support has the formula:
●-P¹—(P²)_{y}—P⁴ I
wherein:
P¹ is an arginine surrogate attached directly to a solid support;
P² is an amino acid residue;
y is an integer from 1 to about 12 wherein each P² can be the same or different; and
P⁴ is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group.

23. A protease modulator having the formula:
P¹—(P²)_{y}—P⁴ Ia
wherein:
P¹ is an arginine surrogate;
P² is an amino acid residue;
y is an integer from 1 to about 12 wherein each P² can be the same or different; and
P⁴ is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group and an optionally substituted alkyl group.

24. The protease modulator of claim 23, wherein said modulator has the formula 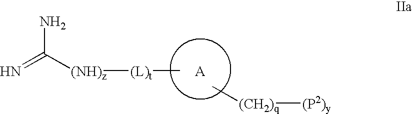
wherein the ring ‘A’ is a member selected from the group consisting of a five- or a six-membered optionally substituted cycloalkyl group, optionally substituted aryl group and an optionally substituted heteroaryl group, wherein said heteroaryl group has from 1 to 3 heteroatoms selected from the group consisting of optionally substituted N, O, and S; and
z is 0 or 1;
L is selected from the group consisting of alkylene optionally interrupted by a heteroatom and S;
t is 0 or 1;
q is an integer from 0 to about 3; and
y is an integer from 1 to about 12 wherein each P² can be the same or different.

25. The protease modulator of claim 23, wherein said modulator has the formula 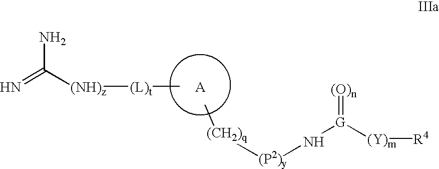
wherein:
the ring ‘A’ is a member selected from the group consisting of a five- or a six-membered optionally substituted cycloalkyl group, optionally substituted aryl group and an optionally substituted heteroaryl group, wherein said heteroaryl group has from 1 to 3 heteroatoms selected from the group consisting of N, O, and S;
each P² may be the same or a different amino acid residue;
y is an integer of 1 to about 3;
G is carbon and n is 1; or alternatively,
G is sulfur and n is 0, 1 or 2;
Y is a heteroatom selected from the group consisting of optionally substituted N and O;
m is 0 or 1; and
R⁴ is an optionally substituted aryl group, an optionally substituted heteroaryl group, an optionally substituted arylalkyl group, an optionally substituted heteroarylalkyl group and an optionally substituted alkyl group.

26. The protease modulator of claim 25, wherein said modulator has the formula 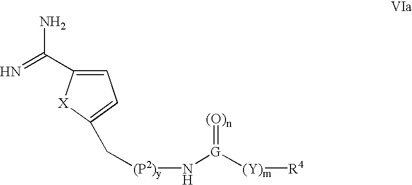
wherein:
X is a heteroatom selected from the group consisting of optionally substituted N, S and O;
each P² may be the same or different amino acid residue;
y is an integer of 1 to about 3;
G is carbon and n is 1; or alternatively,
G is sulfur and n is 0, 1 or 2;
Y is a heteroatom selected from the group consisting of optionally substituted N and O;
m is 0 or 1; and
R⁴ is an optionally substituted aryl group, an optionally substituted heteroaryl group, an optionally substituted arylalkyl group, an optionally substituted heteroarylalkyl group and an optionally substituted alkyl group.

27. A method for modulating a protease, said method comprising:
contacting said protease with a compound having the formula:
P¹—(P²)_{y}—P⁴ Ia
wherein:
P¹ is an arginine surrogate;
P² is an amino acid residue;
y is an integer from 1 to about 12 wherein each P² can be the same or different; and
P⁴ is a member selected from the group consisting of R—, RC(O)—, RR¹NC(O)—, RSO₂—, RR¹N—SO₂—, RS(O)—, RR¹N—S(O)— and ROC(O)—, wherein R and R¹ are each independently selected from the group consisting of hydrogen, an optionally substituted aryl group, an optionally substituted heteroaryl group, an optionally substituted arylalkyl group, an optionally substituted heteroarylalkyl group, an optionally substituted heteroarylaryl group, an optionally substituted cycloalkyl group, an optionally substituted alkoxy group and an optionally substituted alkyl group, thereby modulating said protease.

28. The method for modulating a protease of claim 27, wherein said protease is a serine protease.

29. The method for modulating a protease of claim 28, wherein said serine protease is a Type II membrane bound protease.

30. An amidine derivative composition, said amidine derivative composition comprising:
a) an amidine derivative attached directly to a solid support having the formula: 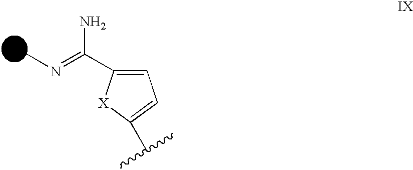
wherein X is a heteroatom selected from the group consisting of optionally substituted N, S and O; and
b) a solid support
